SEQUENCE LISTING (64 sequences)

SEQ ID NO: 1 - DNA, 32 bp, synthetic construct
  source 1..32: mol_type = other DNA
  modified_base 32: mod_base = OTHER; note = t-butylbenzyl-dA
gactgcgatataggattaatcattatagaagn

SEQ ID NO: 2 - DNA, 21 bp, synthetic construct
  source 1..21: mol_type = other DNA
  modified_base 21: mod_base = OTHER; note = t-butylbenzyl-dA
gatggtcctcccagattccgn

SEQ ID NO: 3 - DNA, 30 bp, synthetic construct
  source 1..30: mol_type = other DNA
  misc_feature 1: note = quencher attached to a nucleotide sequence
  misc_feature 30: note = spacer modified nucleotide
gattaaccttagcagtatcctgagtacggc

SEQ ID NO: 4 - DNA, 29 bp, synthetic construct
  source 1..29: mol_type = other DNA
  modified_base 29: mod_base = OTHER; note = t-butylbenzyl-dA
tgcattgttattttcaccagctgtattan

SEQ ID NO: 5 - DNA, 24 bp, synthetic construct
  source 1..24: mol_type = other DNA
  modified_base 24: mod_base = OTHER; note = t-butylbenzyl-dC
ccatttgctgggcttgattattan

SEQ ID NO: 6 - DNA, 30 bp, synthetic construct
  source 1..30: mol_type = other DNA
  misc_feature 1: note = quencher attached to a nucleotide sequence
  misc_feature 30: note = spacer modified nucleotide
atcaagtgacaactccacaagtggtaaatc

SEQ ID NO: 7 - DNA, 32 bp, synthetic construct
  source 1..32: mol_type = other DNA
  modified_base 32: mod_base = OTHER; note = t-butylbenzyl-dA
cccgaaaaatacgcaaaatttcattacatctn

SEQ ID NO: 8 - DNA, 30 bp, synthetic construct
  source 1..30: mol_type = other DNA
  modified_base 30: mod_base = OTHER; note = t-butylbenzyl-dC
cgtggaatgacataaaccttcatcaatctn

SEQ ID NO: 9 - DNA, 34 bp, Synthetic construct
  source 1..34: mol_type = other DNA
  misc_feature 1: note = quencher attached to a nucleotide sequence
  misc_feature 34: note = spacer modified nucleotide
caggaaaaggacattttaccaaggaacttgtgga

SEQ ID NO: 10 - DNA, 31 bp, synthetic construct
  source 1..31: mol_type = other DNA
  modified_base 31: mod_base = OTHER; note = t-butylbenzyl-dA
ttccttaccatttaagcacacaaattattan

SEQ ID NO: 11 - DNA, 21 bp, Synthetic construct
  source 1..21: mol_type = other DNA
  modified_base 21: mod_base = OTHER; note = t-butylbenzyl-dA
ggcagcttaagcaattgctgn

SEQ ID NO: 12 - DNA, 27 bp, synthetic construct
  source 1..27: mol_type = other DNA
  misc_feature 1: note = quencher attached to a nucleotide sequence
  misc_feature 27: note = spacer modified nucleotide
gatattcaccgaacactagggttgctc

SEQ ID NO: 13 - DNA, 35 bp, synthetic construct
  source 1..35: mol_type = other DNA
  modified_base 35: mod_base = OTHER; note = t-butylbenzyl-dC
tataaaagatagtcaaaactttattacttcaaagn

SEQ ID NO: 14 - DNA, 27 bp, synthetic construct
  source 1..27: mol_type = other DNA
  modified_base 27: mod_base = OTHER; note = t-butylbenzyl-dA
ggatctatttcaatggcggttacatan

SEQ ID NO: 15 - DNA, 28 bp, synthetic construct
  source 1..28: mol_type = other DNA
  misc_feature 1: note = quencher attached to a nucleotide sequence
  misc_feature 28: note = spacer modified nucleotide
ttggttcagggaaaggtcatttcacgtt

SEQ ID NO: 16 - DNA, 34 bp, synthetic construct
  source 1..34: mol_type = other DNA
  modified_base 34: mod_base = OTHER; note = t-butylbenzyl-dC
agaattatttgaaatggatatgaagaactatagn

SEQ ID NO: 17 - DNA, 34 bp, synthetic construct
  source 1..34: mol_type = other DNA
  modified_base 34: mod_base = OTHER; note = t-butylbenzyl-dA
tcttttataatttcctcaatctgtattcttgatn

SEQ ID NO: 18 - DNA, 35 bp, synthetic construct
  source 1..35: mol_type = other DNA
  misc_feature 1: note = quencher attached to a nucleotide sequence
  misc_feature 35: note = spacer modified nucleotide
gttttaattgctgtaagtttgtcaaaggcagctca

SEQ ID NO: 19 - DNA, 30 bp, synthetic construct
  source 1..30: mol_type = other DNA
  modified_base 30: mod_base = OTHER; note = t-butylbenzyl-dA
tcaagaagatgctttatatcgtccgtttan

SEQ ID NO: 20 - DNA, 28 bp, synthetic construct
  source 1..28: mol_type = other DNA
  modified_base 28: mod_base = OTHER; note = t-butylbenzyl-dC
aggttcatcaataagcaggaaacaactn

SEQ ID NO: 21 - DNA, 24 bp, synthetic construct
  source 1..24: mol_type = other DNA
  misc_feature 1: note = quencher attached to a nucleotide sequence
  misc_feature 24: note = spacer modified nucleotide
caaacgaaggtccttcttgcagct

SEQ ID NO: 22 - DNA, 27 bp, synthetic construct
  source 1..27: mol_type = other DNA
  modified_base 26: mod_base = OTHER; note = 2'-OMethyl-riboU
ggggttgtaggactgcgatataggant

SEQ ID NO: 23 - DNA, 27 bp, synthetic construct
  source 1..27: mol_type = other DNA
  modified_base 2: mod_base = OTHER; note = 7-deaza-dG
  modified_base 26: mod_base = OTHER; note = 2-O-Methyl-rU
gnggttgtaggactgcgatataggant

SEQ ID NO: 24 - DNA, 33 bp, synthetic construct
  source 1..33: mol_type = other DNA
  modified_base 33: mod_base = OTHER; note = t-butylbenzyl-dA
cgtactcaggatactgctaaggttaatctatcn

SEQ ID NO: 25 - DNA, 43 bp, synthetic construct
  source 1..43: mol_type = other DNA
  misc_feature 1: note = quencher attached to a nucleotide sequence
  misc_feature 43: note = C3 Spacer modified nucleotide
attatagaagaattacctgggaaggtaagccaaagagagtaac

SEQ ID NO: 26 - DNA, 42 bp, synthetic construct
  source 1..42: mol_type = other DNA
  misc_feature 1: note = quencher attached to a nucleotide sequence
  misc_feature 42: note = C3 Spacer modified nucleotide
ttatagaagaattacctgggaaggtaagccaaagagagtaac

SEQ ID NO: 27 - DNA, 41 bp, synthetic construct
  source 1..41: mol_type = other DNA
  misc_feature 1: note = quencher attached to a nucleotide sequence
  misc_feature 41: note = C3 Spacer modified nucleotide
tatagaagaattacctgggaaggtaagccaaagagagtaac

SEQ ID NO: 28 - DNA, 31 bp, Synthetic construct
  source 1..31: mol_type = other DNA
  modified_base 31: mod_base = OTHER; note = t-butylbenzyl-dA
tgcattgttattttcaccagctgtattagan

SEQ ID NO: 29 - DNA, 30 bp, Synthetic construct
  source 1..30: mol_type = other DNA
  modified_base 30: mod_base = OTHER; note = t-butylbenzyl-dA
ttttgagccatttgctgggcttgattattn

SEQ ID NO: 30 - DNA, 29 bp, Synthetic construct
  source 1..29: mol_type = other DNA
  misc_feature 1: note = quencher attached to a nucleotide sequence
  misc_feature 29: note = C3 Spacer modified nucleotide
tcaagtgacaactccacaagtggtaaatc

SEQ ID NO: 31 - DNA, 29 bp, Synthetic construct
  source 1..29: mol_type = other DNA
  modified_base 29: mod_base = OTHER; note = t-butylbenzyl-dC
attgttttctccagcgattttggaagttk

SEQ ID NO: 32 - DNA, 35 bp, Synthetic construct
  source 1..35: mol_type = other DNA
  modified_base 35: mod_base = OTHER; note = t-butylbenzyl-dC
tcaagtttttgttttatttgttgttgattattaan

SEQ ID NO: 33 - DNA, 31 bp, Synthetic construct
  source 1..31: mol_type = other DNA
  modified_base 31: mod_base = OTHER; note = t-butylbenzyl-dA
aaaattgttgggaatattccttaccatttan

SEQ ID NO: 34 - DNA, 23 bp, Synthetic construct
  source 1..23: mol_type = other DNA
  modified_base 23: mod_base = OTHER; note = t-butylbenzyl-dA
gcaattgctgaatcgagacttgn

SEQ ID NO: 35 - DNA, 11 bp, Synthetic construct
  source 1..11: mol_type = other DNA
  misc_feature 1: note = fluorescent dye modified nucleotide
  modified_base 2: mod_base = OTHER; note = D_LNA_A
  misc_feature 11: note = quencher attached to a nucleotide sequence
tnccttggata

SEQ ID NO: 36 - DNA, 23 bp, Synthetic construct
  source 1..23: mol_type = other DNA
  misc_feature 1: note = quencher attached to a nucleotide sequence
  misc_feature 23: note = C3 Spacer modified nucleotide
ttcaccgaacactagggttgctc

SEQ ID NO: 37 - DNA, 28 bp, Synthetic construct
  source 1..28: mol_type = other DNA
  misc_feature 1: note = quencher attached to a nucleotide sequence
  misc_feature 28: note = C3 Spacer modified nucleotide
ttggttcagggaaaggtcatttcacgtt

SEQ ID NO: 38 - DNA, 35 bp, Synthetic construct
  source 1..35: mol_type = other DNA
  modified_base 35: mod_base = OTHER; note = t-butylbenzyl-dA
tcattacatctaaaaagcatgtaaaggaaatattn

SEQ ID NO: 39 - DNA, 33 bp, Synthetic construct
  source 1..33: mol_type = other DNA
  modified_base 33: mod_base = OTHER; note = t-butylbenzyl-dA
gtggaatgacataaaccttcatcaatctctatn

SEQ ID NO: 40 - DNA, 10 bp, Synthetic construct
  source 1..10: mol_type = other DNA
  misc_feature 1: note = fluorescent dye modified nucleotide
  misc_feature 10: note = quencher attached to a nucleotide sequence
ttgggtcagg

SEQ ID NO: 41 - DNA, 30 bp, Synthetic construct
  source 1..30: mol_type = other DNA
  misc_feature 1: note = quencher attached to a nucleotide sequence
  misc_feature 30: note = C3 Spacer modified nucleotide
aaaaggacattttaccaaggaacttgtgga

SEQ ID NO: 42 - DNA, 35 bp, Synthetic construct
  source 1..35: mol_type = other DNA
  modified_base 35: mod_base = OTHER; note = t-butylbenzyl-dC
ggtgatgtaaaattagcaagtaatttaaaaatatn

SEQ ID NO: 43 - DNA, 31 bp, Synthetic construct
  source 1..31: mol_type = other DNA
  modified_base 31: mod_base = OTHER; note = t-butylbenzyl-dA
tcgctatagttcttcatatccatttcaaatn

SEQ ID NO: 44 - DNA, 36 bp, Synthetic construct
  source 1..36: mol_type = other DNA
  misc_feature 1: note = quencher attached to a nucleotide sequence
  misc_feature 36: note = C3 Spacer modified nucleotide
gtgttgatgaaacattgtgcaaaacaattcttagaa

SEQ ID NO: 45 - DNA, 27 bp, Synthetic construct
  source 1..27: mol_type = other DNA
  misc_feature 1: note = quencher attached to a nucleotide sequence
  misc_feature 27: note = C3 Spacer modified nucleotide
gagcaaacgaaggtccttcttgcagct

SEQ ID NO: 46 - DNA, 27 bp, Synthetic construct
  source 1..27: mol_type = other DNA
  misc_feature 1: note = quencher attached to a nucleotide sequence
  misc_feature 27: note = C3 Spacer modified nucleotide
atagaagaattacctgggaaggtaagc

SEQ ID NO: 47 - DNA, 27 bp, synthetic construct
  source 1..27: mol_type = other DNA
  misc_feature 1: note = quencher attached to a nucleotide sequence
  misc_feature 27: note = C3 Spacer modified nucleotide
atagaagaattacctgggaaggtaagc

SEQ ID NO: 48 - DNA, 26 bp, Synthetic construct
  source 1..26: mol_type = other DNA
  misc_feature 1: note = quencher attached to a nucleotide sequence
  misc_feature 26: note = C3 Spacer modified nucleotide
tagaagaattacctgggaaggtaagc

SEQ ID NO: 49 - DNA, 26 bp, Synthetic construct
  source 1..26: mol_type = other DNA
  misc_feature 1: note = quencher attached to a nucleotide sequence
  misc_feature 26: note = C3 Spacer modified nucleotide
tagaagaattacctgggaaggtaagc

SEQ ID NO: 50 - DNA, 29 bp, Synthetic construct
  source 1..29: mol_type = other DNA
  misc_feature 1: note = quencher attached to a nucleotide sequence
  misc_feature 29: note = C3 Spacer modified nucleotide
ttatagaagaattacctgggaaggtaagc

SEQ ID NO: 51 - DNA, 29 bp, Synthetic construct
  source 1..29: mol_type = other DNA
  misc_feature 1: note = quencher attached to a nucleotide sequence
  misc_feature 29: note = C3 Spacer modified nucleotide
ttatagaagaattacctgggaaggtaagc

SEQ ID NO: 52 - DNA, 27 bp, Synthetic construct
  source 1..27: mol_type = other DNA
  misc_feature 1: note = quencher attached to a nucleotide sequence
  misc_feature 27: note = C3 Spacer modified nucleotide
atagaagaattacctgggaaggtaagc

SEQ ID NO: 53 - DNA, 27 bp, Synthetic construct
  source 1..27: mol_type = other DNA
  misc_feature 1: note = quencher attached to a nucleotide sequence
  misc_feature 27: note = C3 Spacer modified nucleotide
atagaagaattacctgggaaggtaagc

SEQ ID NO: 54 - DNA, 26 bp, Synthetic construct
  source 1..26: mol_type = other DNA
  misc_feature 1: note = quencher attached to a nucleotide sequence
  misc_feature 26: note = C3 Spacer modified nucleotide
tagaagaattacctgggaaggtaagc

SEQ ID NO: 55 - DNA, 26 bp, Synthetic construct
  source 1..26: mol_type = other DNA
  misc_feature 1: note = quencher attached to a nucleotide sequence
  misc_feature 26: note = C3 Spacer modified nucleotide
tagaagaattacctgggaaggtaagc

SEQ ID NO: 56 - DNA, 29 bp, Synthetic construct
  source 1..29: mol_type = other DNA
  misc_feature 1: note = quencher attached to a nucleotide sequence
  misc_feature 29: note = C3 Spacer modified nucleotide
ttatagaagaattacctgggaaggtaagc

SEQ ID NO: 57 - DNA, 29 bp, Synthetic construct
  source 1..29: mol_type = other DNA
  misc_feature 1: note = quencher attached to a nucleotide sequence
  misc_feature 29: note = C3 Spacer modified nucleotide
ttatagaagaattacctgggaaggtaagc

SEQ ID NO: 58 - DNA, 42 bp, Synthetic construct; identical to SEQ ID NO: 26
  source 1..42: mol_type = other DNA
  misc_feature 1: note = quencher attached to a nucleotide sequence
  misc_feature 42: note = C3 Spacer modified nucleotide

SEQ ID NO: 59 - DNA, 42 bp, Synthetic construct; identical to SEQ ID NO: 26
  source 1..42: mol_type = other DNA
  misc_feature 1: note = quencher attached to a nucleotide sequence
  misc_feature 42: note = C3 Spacer modified nucleotide

SEQ ID NO: 60 - DNA, 27 bp, Synthetic construct
  source 1..27: mol_type = other DNA
  misc_feature 1: note = quencher attached to a nucleotide sequence
  misc_feature 27: note = C3 Spacer modified nucleotide
aagtgacaactccacaagtggtaaatc

SEQ ID NO: 61 - DNA, 26 bp, Synthetic construct
  source 1..26: mol_type = other DNA
  misc_feature 1: note = quencher attached to a nucleotide sequence
  misc_feature 26: note = C3 Spacer modified nucleotide
agtgacaactccacaagtggtaaatc

SEQ ID NO: 62 - DNA, 27 bp, Synthetic construct
  source 1..27: mol_type = other DNA
  misc_feature 1: note = quencher attached to a nucleotide sequence
  misc_feature 27: note = C3 Spacer modified nucleotide
aagctactaatcatgtcgtagttagtc

SEQ ID NO: 63 - DNA, 26 bp, Synthetic construct
  source 1..26: mol_type = other DNA
  misc_feature 1: note = quencher attached to a nucleotide sequence
  misc_feature 26: note = C3 Spacer modified nucleotide
agctactaatcatgtcgtagttagtc

SEQ ID NO: 64 - DNA, 30 bp, Synthetic construct
  source 1..30: mol_type = other DNA
  misc_feature 1: note = quencher attached to a nucleotide sequence
  misc_feature 30: note = C3 Spacer modified nucleotide
atcaagctactaatcatgtcgtagttagtc

(19) United States
(12) Patent Application Publication
(10) Pub. No.: US 2026/0250784 A1
(43) Pub. Date: Aug. 27, 2026

(54) COMPOSITIONS AND METHODS FOR THE DETECTION OF GROUP B STREPTOCOCCUS (STREPTOCOCCUS AGALACTIAE) AND CLINDAMYCIN RESISTANCE GENE DETERMINANTS

(71) Applicant: Roche Molecular Systems, Inc, Pleasanton, CA (US)

(72) Inventors: Jeffrey Alexander, Hayward, CA (US); Kyle C. Cady, Redwood City, CA (US); Ke Chen, Palo Alto, CA (US); Letong Jia, San Jose, CA (US); Colin Lam, Redwood City, CA (US); Marc Rehfuss, Alameda, CA (US); Jingtao Sun, San Ramon, CA (US); Xun Zhuang, Pleasanton, CA (US)

(21) Appl. No.: 19/161,689

(22) PCT Filed: Feb. 29, 2024
(86) PCT No.: PCT/EP2024/055178

§ 371 Date: Sep. 2, 2025

Related U.S. Application Data
(60) Provisional application No. 63/488,446, filed on Mar. 3, 2023.

(51) Int. Cl. C12Q 1/689; C12Q 1/6809; C12Q 1/6848; C12Q 1/686

(52) U.S. Cl. CPC C12Q 1/689; C12Q 1/6809; C12Q 1/6848; C12Q 1/686; C12Q 2600/158; C12Q 2600/16

(57) ABSTRACT

The present invention relates to compositions and methods to detect Group B Streptococcus (GBS, Streptococcus agalactiae) and to identify the most prevalent genes responsible for clindamycin resistant GBS such as ermB, ermTR, ermT, lsaC and lsaE by a multiplex real-time PCR assay.

### CROSS REFERENCE TO RELATED APPLICATIONS

This application is based on and claims priority from U.S. Provisional Patent Application No. 63/488,446, filed Mar. 3, 2023, which is incorporated by reference herein in its entirety.

### REFERENCE SEQUENCE LISTING

This application contains a Sequence Listing submitted as an electronic text file named “P38225-WO PCT Seq_Listing”, having a size in bytes of 24,942 bytes, and created on Feb. 13, 2024. The information contained in this electronic file is hereby incorporated by reference in its entirety pursuant to 37 CFR § 1.52(e)(5).

### FIELD OF THE INVENTION

The present disclosure relates to the field of bacterial diagnostics, and more particularly to the detection of Group B Streptococcus that are resistant to the antibiotic clindamycin.

### BACKGROUND OF THE INVENTION

Group B Streptococcal infection has been a leading cause of neonatal infection, caused by the bacterium Streptococcus agalactiae (also known as group B streptococcus—GBS). GBS infection in the first week of life, defined as early-onset disease (EOD), can cause serious illness such as sepsis, meningitis, pneumonia or even death (Schuchat, Anne, “Group B streptococcus”, The Lancet 353.9146 (1999): 51-56). GBS consists of a single species, Streptococcus agalactiae which is a gram positive commensal bacteria found in the enteric and reproductive tracts. Bacterial transmission can occur during labor as the fetus passes through the birth canal of a colonized mother. Prevention of GBS EOD through universal screening during 36th and 38th week of pregnancy along with antibiotic prophylaxis heavily reduces the chance of disease (“Prevention of Group B Streptococcal Early-Onset Disease in Newborns ACOG COMMITTEE OPINION Number 797”, Obstetrics & Gynecology, 135 (2)(2020): e51-72). The Centers for Disease Control and Prevention (CDC) previously provided guidelines for stewardship, but in 2018 responsibility shifted to three professional organizations. The American College of Obstetricians and Gynecologists and American Academy of Pediatrics provide guidelines on prophylaxis and treatment, while American Society of Microbiology (ASM) provides guidelines for standard clinical laboratory practices. (American College of Obstetricians and Gynecologists Committee on Obstetric Practice. “Prevention of early-onset group B streptococcal disease in newborns: ACOG committee opinion no 782”, Obstet Gynecol 134 (2019): e19-e40).

According to ASM guidelines, a vaginal-rectal swab is taken from a patient and placed into overnight enrichment. It can then be subjected to plate based agar for identification or nucleic acid amplification test for GBS identification. A positive GBS colonization result leads to a recommendation of intravenous penicillin prophylaxis. If a patient is reported to having a penicillin allergy with low risk of anaphylaxis, a first generation cephalosporin is recommended. In cases of patients with penicillin allergy and a high risk of anaphylaxis, clindamycin is recommended.

GBS has been found to be increasingly resistant to clindamycin, with up to 40% of isolates in certain regions (CDC. “Antibiotic Resistance Threats in the United States”. 2019). Due to the high rate of resistance, antibiotic susceptibility test is carried out, which are typically culture based and can add several days for a result. Constitutive and inducible clindamycin resistance can be attributed to five different resistance genes: the 23S rRNA methylase genes, ermB, ermTR, ermT, and the ATP-binding cassette (ABC) transport genes, lsaC and lsaE. ermB and ermTR has been shown to account for almost 90% of resistant isolates in the United States. (Metcalf, B. J., et al. “Short-read whole genome sequencing for determination of antimicrobial resistance mechanisms and capsular serotypes of current invasive Streptococcus agalactiae recovered in the USA”, Clinical Microbiology and Infection 23.8 (2017): 574-e7). There are no current molecular assays to rapidly determine GBS clindamycin resistance.

Multiple on-market molecular assays exist for GBS screening, but most require an overnight enrichment culture before usage. Direct from sample testing can drastically reduce time to result, but better sensitivity and specificity are needed for robust detection of GBS (Filkins, L., et al. “Guidelines for the detection and identification of group B streptococcus.” Am Soc Microbiol). Furthermore, currently there are no known on-market assays for GBS and clindamycin resistance testing direct from sample or from enriched culture. A similar direct from sample assay was attempted (Gygax et al. “Detection of erythromycin and clindamycin resistance genes in Group B Streptococcal clinical isolates and cervicovaginal-rectal swabs”, Microb Drug Resist., 2007 Summer; 13(2):119-23), but had difficulty discerning if the antibiotic resistance genes were from GBS, when the genes were present in other Gram-positive bacteria contained in the sample.

### SUMMARY OF THE INVENTION

The present invention discloses a multiplex PCR assay that utilizes dual GBS targets: a single-copy target and a multi-copy target that allows for a decreased Limit of Detection (LoD) for a direct from sample workflow. The assay also adds oligonucleotide primer/oligonucleotide probe combinations to detect the five genes responsible for clindamycin resistance, which allows rapid clindamycin resistance information. Using the dual GBS targets allows for the determination of whether the clindamycin-resistance genes are from GBS or not. In one aspect, an algorithm for this determination is based on the difference in Ct values between a single-copy GBS gene and at least one clindamycin resistance genes.

Provided herein are methods for the rapid detection of the presence or absence of Group B Streptococcus (GBS, Streptococcus agalactiae or S. agalactiae) and the most prevalent clindamycin resistance genes in GBS in a biological or non-biological sample. This is accomplished, for example, by multiplex detection of the multi-copy 23S ribosomal RNA (23S rRNA) gene and the single-copy cAMP factor (cfb) gene of GBS, and also the ermTR, ermB, ermT, lsaC and lsaE genes that confer resistance to clindamycin by real-time polymerase chain reaction in a single test tube. Hence, methods of detection of the 23S rRNA and cfb genes and the clindamycin resistance genes are provided comprising performing at least one cycling step, which may include an amplifying step and a hybridizing step. Furthermore, provided are oligonucleotide primers, oligonucleotide probes, and kits that are designed for the detection of the GBS 23S rRNA gene, the GBS cfb gene and the clindamycin resistance genes, ermTR, ermB, ermT, lsaC and lsaE, in a single tube. The detection methods are designed to target these genes, which allows one to detect the presence of GBS and the mechanism of clindamycin resistance in a single test.

Provided herein is a method for detecting GBS having clindamycin resistance mechanisms in a sample, including performing an amplifying step including contacting the sample with a set of GBS 23SrRNA forward and reverse oligonucleotide primers, a set of GBS cfb forward and reverse oligonucleotide primers, a set of ermTR forward and reverse oligonucleotide primers, a set of ermB forward and reverse oligonucleotide primers, a set of ermT forward and reverse oligonucleotide primers, a set of lsaC forward and reverse oligonucleotide primers and a set of lsaE forward and reverse oligonucleotide primers to produce amplification product(s) if any of these target genes are present in the sample; performing a hybridizing step including contacting the amplification product(s) with one or more detectable GBS 23S rRNA oligonucleotide probes, one or more detectable GBS cfb oligonucleotide probes, one or more detectable ermTR oligonucleotide probes, one or more detectable ermB oligonucleotide probes, one or more detectable ermT oligonucleotide probes, one or more detectable lsaC oligonucleotide probes, and one or more detectable lsaE oligonucleotide probes; and detecting the presence or absence of the amplification product(s), wherein the presence of the amplification product(s) is indicative of the presence of GBS and/or a clindamycin resistance mechanism in the sample and wherein the absence of the amplification product(s) is indicative of the absence of GBS and/or a clindamycin resistance mechanism in the sample.

In one aspect, a method for detecting Group B Streptococcus (GBS) is provided, the method comprising: contacting a sample with a plurality of oligonucleotide primers for a defined set of targets to produce an amplification product comprising a representative nucleic acid for each of the targets present in the sample; combining the amplification product with a detectable oligonucleotide probe for each of the targets; and detecting the presence or absence of each of the representative nucleic acids in the amplification product, wherein the presence or absence of each of the representative nucleic acids in the amplification product is indicative of the presence or absence in the sample of each of a GBS strain and a clindamycin resistance mechanism. In one embodiment, the method further distinguishes GBS from other species of Streptococcus. In one embodiment, the sample is selected from one of an enriched sample and a direct from specimen sample. In one embodiment, the defined set of targets comprises: i) a GBS 23s ribosomal RNA gene (23s rRNA), ii) a GBS-specific gene, and iii) at least one clindamycin resistance gene. In one embodiment, the method further comprises: measuring a cycle threshold (Ct) for each of the GBS-specific gene (Ct_{GBS}) and the at least one clindamycin resistance gene (Ct_{ClinR}); calculating the difference between Ct_{GBS} and Ct_{ClinR} as ΔCt; and identifying the sample as comprising GBS harboring the at least one clindamycin resistance gene when the absolute value of ΔCt is less than or equal to a threshold value (x). In one embodiment, x≤2. In one embodiment, the GBS-specific gene is selected from the group consisting of CAMP-factor encoding gene (cfb), surface immunogenic protein encoding gene (sip), a glycosyl transferase protein encoding gene, and a lysR family protein encoding gene. In a certain embodiment, the GBS-specific gene is the CAMP-factor encoding gene (cfb). In one embodiment, the at least one clindamycin resistance gene is selected from the group consisting of ermTR, ermB, ermT, lsaC, and lsaE. In one embodiment, the plurality of oligonucleotide primers comprises a set of oligonucleotide primers for amplifying at least a portion of each of the targets, wherein the set of 23s rRNA oligonucleotide primers comprises at least one primer comprising a nucleic acid sequence of SEQ ID NOs: 22, 23 or 24; the set of GBS-specific gene oligonucleotide primers comprises at least one primer comprising a nucleic acid sequence of SEQ ID NOs: 28, 29, 31, or 32; and the at least one set of clindamycin resistance gene oligonucleotide primers is selected from the group consisting of: a set of ermTR oligonucleotide primers comprising at least one primer comprising a nucleic acid sequence of SEQ ID NOs: 38 or 39; a set of ermB oligonucleotide primers comprising at least one primer comprising a nucleic acid sequence of SEQ ID NOs: 33 or 34; a set of ermT oligonucleotide primers comprising at least one primer comprising a nucleic acid sequence of SEQ ID NOs: 13 or 14, a set of lsaC oligonucleotide primers comprising at least one primer comprising a nucleic acid sequence of SEQ ID NOs: 42 or 43, and a set of lsaE oligonucleotide primers comprising at least one primer comprising a nucleic acid sequence of SEQ ID NOs: 19 or 20. In one embodiment, the detectable oligonucleotide probe for 23s rRNA comprises a nucleic acid sequence of SEQ ID NO: 27 or a complement thereof, the detectable oligonucleotide probe for the GBS-specific gene comprises a nucleic acid sequence of SEQ ID NO: 30 or 64 or a complement thereof, and the detectable oligonucleotide probe for the at least one clindamycin resistance gene is selected from the group consisting of: a detectable oligonucleotide probe for ermTR comprising a nucleic acid sequence of SEQ ID NOs: 40 or 41 or a complement thereof, a detectable oligonucleotide probe for ermB comprising a nucleic acid sequence of SEQ ID NOs: 35 or 36 or a complement thereof, a detectable oligonucleotide probe for ermT comprising a nucleic acid sequence of SEQ ID NO: 37 or a complement thereof, a detectable oligonucleotide probe for lsaC comprising a nucleic acid sequence of SEQ ID NO: 44 or a complement thereof, and a detectable oligonucleotide probe for lsaE comprising a nucleic acid sequence of SEQ ID NO: 45 or a complement thereof. In some embodiments, the detectable oligonucleotide probe for each of the targets is labeled with a donor moiety and a corresponding acceptor moiety, and wherein the detecting step further comprises detecting the presence or absence of fluorescence resonance energy transfer (FRET) between the donor moiety and the acceptor moiety of the detectable oligonucleotide probe, wherein the presence or absence of a fluorescence signal from the detectable oligonucleotide probe is indicative of the presence or absence of a corresponding one of the targets in the sample. In some embodiments, the donor moiety and the corresponding acceptor moiety are separated by at least 7 nucleotides on the detectable oligonucleotide probe. In one embodiment, the acceptor moiety is a quencher. In one embodiment, the contacting step further comprises a polymerase enzyme having 5′ to 3′ nuclease activity.

In one embodiment, the set of GBS 23S rRNA oligonucleotide primers comprises or consists of a forward primer comprising or consisting of a nucleic acid sequence of SEQ ID NO: 22 or 23 and/or a reverse primer comprising or consisting of a nucleic acid sequence of SEQ ID NO: 24 and/or the detectable GBS 23SrRNA oligonucleotide probe comprises or consists of a nucleic acid sequence of SEQ ID NO: 27, or complement thereof. In one embodiment, the set of GBS cfb oligonucleotide primers comprises or consists of a forward primer comprising or consisting of a nucleic acid sequence of SEQ ID NO: 28 or 31 and/or a reverse primer comprising or consisting of a nucleic acid sequence of SEQ ID NO: 29 or 32 and/or the detectable GBS cfb oligonucleotide probe comprises or consists of a nucleic acid sequence of SEQ ID NO: 30 or 64, or complement thereof. In one embodiment, the set of ermTR oligonucleotide primers comprises or consists of a forward primer comprising or consisting of a nucleic acid sequence of SEQ ID NO: 38 and/or a reverse primer comprising or consisting of a nucleic acid sequence of SEQ ID NO: 39 and/or the detectable ermTR oligonucleotide probe comprises or consists of a nucleic acid sequence of SEQ ID NO: 40 or 41, or complement thereof. In one embodiment, the set of ermB oligonucleotide primers comprises or consists of a forward primer comprising or consisting of a nucleic acid sequence of SEQ ID NO: 33 and/or a reverse primer comprising or consisting of a nucleic acid sequence of SEQ ID NO: 34 and/or the detectable ermB oligonucleotide probe comprises or consists of a nucleic acid sequence of SEQ ID NO: 35 or 36, or complement thereof. In one embodiment, the set of ermT oligonucleotide primers comprises or consists of a forward primer comprising or consisting of a nucleic acid sequence of SEQ ID NO: 13 and/or a reverse primer comprising or consisting of a nucleic acid sequence of SEQ ID NO: 14 and/or the detectable ermT oligonucleotide probe comprises or consists of a nucleic acid sequence of SEQ ID NO: 37, or complement thereof. In one embodiment, the set of lsaC oligonucleotide primers comprises or consists of a forward primer comprising or consisting of a nucleic acid sequence of SEQ ID NO: 42 and/or a reverse primer comprising or consisting of a nucleic acid sequence of SEQ ID NO: 43 and/or the detectable lsaC oligonucleotide probe comprises or consists of a nucleic acid sequence of SEQ ID NO: 44, or complement thereof. In one embodiment, the set of lsaE oligonucleotide primers comprises or consists of a forward primer comprising or consisting of a nucleic acid sequence of SEQ ID NO: 19 and/or a reverse primer comprising or consisting of a nucleic acid sequence of SEQ ID NO: 20 and/or the detectable lsaE oligonucleotide probe comprises or consists of a nucleic acid sequence of SEQ ID NO: 45, or complement thereof.

In another embodiment, the set of GBS 23S rRNA oligonucleotide primers comprises or consists of a forward primer comprising or consisting of a nucleic acid sequence of SEQ ID NO: 1 and/or a reverse primer comprising or consisting of a nucleic acid sequence of SEQ ID NO: 2 and/or the detectable GBS 23SrRNA oligonucleotide probe comprises or consists of a nucleic acid sequence of SEQ ID NO: 3, or complement thereof. In one embodiment, the set of GBS cfb oligonucleotide primers comprises or consists of a forward primer comprising or consisting of a nucleic acid sequence of SEQ ID NO: 4 and/or a reverse primer comprising or consisting of a nucleic acid sequence of SEQ ID NO: 5 and/or the detectable GBS cfb oligonucleotide probe comprises or consists of a nucleic acid sequence of SEQ ID NO: 6, or complement thereof. In one embodiment, the set of ermTR oligonucleotide primers comprises or consists of a forward primer comprising or consisting of a nucleic acid sequence of SEQ ID NO: 7 and/or a reverse primer comprising or consisting of a nucleic acid sequence of SEQ ID NO: 8 and/or the detectable ermTR oligonucleotide probe comprises or consists of a nucleic acid sequence of SEQ ID NO: 9, or complement thereof. In one embodiment, the set of ermB oligonucleotide primers comprises or consists of a forward primer comprising or consisting of a nucleic acid sequence of SEQ ID NO: 10 and/or a reverse primer comprising or consisting of a nucleic acid sequence of SEQ ID NO: 11 and/or the detectable ermB oligonucleotide probe comprises or consists of a nucleic acid sequence of SEQ ID NO: 12, or complement thereof. In one embodiment, the set of ermT oligonucleotide primers comprises or consists of a forward primer comprising or consisting of a nucleic acid sequence of SEQ ID NO: 13 and/or a reverse primer comprising or consisting of a nucleic acid sequence of SEQ ID NO: 14 and/or the detectable ermT oligonucleotide probe comprises or consists of a nucleic acid sequence of SEQ ID NO: 15, or complement thereof. In one embodiment, the set of lsaC oligonucleotide primers comprises or consists of a forward primer comprising or consisting of a nucleic acid sequence of SEQ ID NO: 16 and/or a reverse primer comprising or consisting of a nucleic acid sequence of SEQ ID NO: 17 and/or the detectable lsaC oligonucleotide probe comprises or consists of a nucleic acid sequence of SEQ ID NO: 18, or complement thereof. In one embodiment, the set of lsaE oligonucleotide primers comprises or consists of a forward primer comprising or consisting of a nucleic acid sequence of SEQ ID NO: 19 and/or a reverse primer comprising or consisting of a nucleic acid sequence of SEQ ID NO: 20 and/or the detectable lsaE oligonucleotide probe comprises or consists of a nucleic acid sequence of SEQ ID NO: 21, or complement thereof.

In another aspect, a method for detecting Group B Streptococcus (GBS) is provided, the method comprising: contacting a sample with a plurality of oligonucleotide primers for a defined set of targets to produce an amplification product comprising a representative nucleic acid for each of the targets present in the sample; combining the amplification product with a detectable oligonucleotide probe for each of the targets; and detecting the presence or absence of each of the representative nucleic acids in the amplification product, wherein the presence or absence of each of the representative nucleic acids in the amplification product is indicative of the presence or absence in the sample of a GBS strain, and wherein the method distinguishes GBS from other species of Streptococcus. In one embodiment, the sample is selected from one of an enriched sample and a direct from specimen sample. In one embodiment, the defined set of targets comprises a GBS 23s ribosomal RNA gene (23s rRNA). In one embodiment, the amplification product is further combined with a blocking oligonucleotide. In one embodiment, the defined set of targets further comprises a GBS-specific gene. In one embodiment, the GBS-specific gene is the CAMP-factor encoding gene (cfb). In one embodiment, the method distinguishes GBS from at least one of Streptococcus urinalis, Streptococcus thermophilus, and Streptococcus anginosus. In one embodiment, the defined set of targets further comprises at least one clindamycin resistance gene. In some embodiments, the at least one clindamycin resistance gene is selected from the group consisting of ermTR, ermB, ermT, lsaC, and lsaE. In one embodiment, the above described oligonucleotide primers and/or probes or any of the sets of oligonucleotide primers and/or probes may be used.

In one embodiment, amplification can employ a polymerase enzyme having 5′ to 3′ nuclease activity. Thus, the first and second fluorescent moieties may be within no more than 8 nucleotides of each other along the length of the oligonucleotide probe. In another aspect, the 23S rRNA, cfb, ermTR, ermB, ermT, lsaC and lsaE oligonucleotide probes include a nucleic acid sequence that permits secondary structure formation. Such secondary structure formation generally results in spatial proximity between the first and second fluorescent moiety. According to this method, the second fluorescent moiety on the oligonucleotide probe can be a quencher.

In another aspect, the present invention provides an oligonucleotide comprising or consisting of a sequence of nucleotides selected from SEQ ID NOs: 1-64, or a complement thereof, which oligonucleotide has 100 or fewer nucleotides. The present disclosure further provides an oligonucleotide that includes a nucleic acid having at least 70% sequence identity (e.g., at least 75%, 80%, 85%, 90% or 95%, etc.) to one of SEQ ID NOs: 1-64, or a complement thereof, which oligonucleotide has 100 or fewer nucleotides. Generally, these oligonucleotides may be primer nucleic acids, probe nucleic acids, or the like in these embodiments and may be used in any of the methods provided herein. In certain of these embodiments, the oligonucleotides have 40 or fewer nucleotides (e.g. 35 or fewer nucleotides, 30 or fewer nucleotides, etc.) In some embodiments, any one of the oligonucleotides may comprise at least one modified nucleotide, e.g. to alter nucleic acid hybridization stability relative to unmodified nucleotides. Optionally, the oligonucleotides comprise at least one label and/or at least one quencher moiety. The oligonucleotides may include at least one conservatively modified variation. “Conservatively modified variations” or, simply, “conservative variations” of a particular nucleic acid sequence refers to those nucleic acids, which encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid.

In another aspect, a kit for detecting Group B Streptococcus (GBS) and at least one clindamycin resistance mechanism is provided, the kit comprising: a plurality of oligonucleotide primers for a defined set of targets to produce an amplification product comprising a representative nucleic acid for each of the targets present in the sample; wherein the defined set of targets comprises a GBS 23s ribosomal RNA gene (23s rRNA). In one embodiment, the defined set of targets further comprises at least one of a GBS-specific gene and/or a clindamycin resistance gene. In one embodiment, the kit includes a plurality of oligonucleotide primers and oligonucleotide probes comprising a set of GBS 23S rRNA gene oligonucleotide primers specific for amplification of the GBS 23S rRNA gene, and one or more detectable GBS 23SrRNA oligonucleotide probes specific for detection of the GBS 23S rRNA gene amplification products; and at least one set of clindamycin resistance gene oligonucleotide primers selected from the group consisting of: a set of ermTR gene oligonucleotide primers specific for amplification of the ermTR gene, and one or more detectable ermTR oligonucleotide probes specific for detection of the ermTR gene amplification products; a set of ermB gene oligonucleotide primers specific for amplification of the ermB gene, and one or more detectable ermB oligonucleotide probes specific for detection of the ermB gene amplification products; a set of ermT gene oligonucleotide primers specific for amplification of the ermT gene, and one or more detectable ermT oligonucleotide probes specific for detection of the ermT gene amplification products; a set of lsaC gene oligonucleotide primers specific for amplification of the lsaC gene, and one or more detectable lsaC oligonucleotide probes specific for detection of the lsaC gene amplification products; and a set of lsaE gene oligonucleotide primers specific for amplification of the lsaE gene, and one or more detectable lsaE oligonucleotide probes specific for detection of the lsaE gene amplification products. In one embodiment, the defined set of targets further comprises a GBS-specific gene. In one embodiment, the GBS-specific gene is the CAMP-factor encoding gene (cfb).

In one embodiment, the kit further comprises a set of GBS cfb gene oligonucleotide primers specific for amplification of the GBS cfb gene, and one or more detectable GBS cfb oligonucleotide probes specific for detection of the GBS cfb gene amplification products. In one embodiment, the above described oligonucleotide primers and/or probes or any of the sets of oligonucleotide primers and/or probes may be comprised in the kit. In one embodiment, the kit can include oligonucleotide probes already labeled with donor and corresponding acceptor fluorescent moieties, or can include fluorophoric moieties for labeling the oligonucleotide probes. The kit can also include nucleoside triphosphates, nucleic acid polymerase, and buffers necessary for the function of the nucleic acid polymerase. The kit can also include a package insert and instructions for using the oligonucleotide primers, oligonucleotide probes, and fluorophoric moieties to detect the presence or absence of GBS and/or the clindamycin resistance mechanism in a sample.

In another aspect, a method for detecting Group B Streptococcus (GBS) is provided, the method comprising: contacting a sample with a plurality of oligonucleotide primers for a defined set of targets to produce an amplification product comprising a representative nucleic acid for each of the targets present in the sample; combining the amplification product with a detectable oligonucleotide probe for each of the targets; and detecting the presence or absence of each of the representative nucleic acids in the amplification product, wherein the presence or absence of each of the representative nucleic acids in the amplification product is indicative of the presence or absence in the sample of a GBS strain, and wherein the defined set of targets comprises a GBS 23s ribosomal RNA gene (23s rRNA). In one embodiment, the sample is selected from one of an enriched sample and a direct from specimen sample. In one embodiment, the defined set of targets further comprises at least one of a GBS-specific gene and a clindamycin resistance gene. In one embodiment, the method distinguishes GBS from at least one of Streptococcus urinalis, Streptococcus thermophilus, and Streptococcus anginosus. In one embodiment, the plurality of oligonucleotide primers comprises a set of oligonucleotide primers for amplifying at least a portion of each of targets, wherein the set of 23s rRNA oligonucleotide primers comprises at least one primer comprising or consisting of a nucleic acid sequence of SEQ ID NOs: 22, 23 or 24. In one embodiment, the detectable oligonucleotide probe for 23s rRNA comprises or consists of a nucleic acid sequence SEQ ID NO: 27 or its complement. In one embodiment, the above described oligonucleotide primers and/or probes or any of the sets of oligonucleotide primers and/or probes may be used in the method.

In another aspect, a method for detecting Group B Streptococcus (GBS) is provided, the method comprising: contacting a sample with a plurality of oligonucleotide primers for a defined set of targets to produce an amplification product comprising a representative nucleic acid for each of the targets present in the sample; combining the amplification product with a detectable probe for each of the targets; and detecting the presence or absence of each of the representative nucleic acids in the amplification product, wherein the presence or absence of each of the representative nucleic acids in the amplification product is indicative of the presence or absence in the sample of a GBS strain, wherein the sample is a direct from specimen sample, wherein the defined set of targets comprises a GBS 23s ribosomal RNA gene (23s rRNA), and wherein the method distinguishes GBS from other species of Streptococcus. In one embodiment, the above described oligonucleotide primers and/or probes or any of the sets of oligonucleotide primers and/or probes may be used in the method.

Unless otherwise defined, all technical and scientific terms used herein have the same meaning as commonly understood by one of ordinary skill in the art to which this invention belongs. Although methods and materials similar or equivalent to those described herein can be used in the practice or testing of the present subject matter, suitable methods and materials are described below. In addition, the materials, methods, and examples are illustrative only and not intended to be limiting.

All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### BRIEF DESCRIPTION OF THE DRAWINGS

FIG. 1 shows two sets of qPCR amplification curves for detection of GBS 23S rRNA or Streptococcus urinalis 23S rRNA according to the present disclosure. The set of oligonucleotide primers and oligonucleotide probes used in the reaction exclusively amplifies the GBS 23S rRNA target (left), with no amplification observed for the Streptococcus urinalis 23S rRNA (right). Fluorescence signal is plotted as a function of the cycle number for three template concentrations, including 1*10², 1*10³ and 1*10⁴ copies per reaction for either the GBS 23S rRNA template or the S. urinalis 23S rRNA template.

### DETAILED DESCRIPTION OF THE INVENTION

As used herein, the term “amplifying” refers to the process of synthesizing nucleic acid molecules that are complementary to one or both strands of a template nucleic acid molecule (e.g., GBS 23S rRNA gene). Amplifying a nucleic acid molecule typically includes denaturing the template nucleic acid, annealing primers to the template nucleic acid at a temperature that is below the melting temperatures of the primers, and enzymatically elongating from the primers to generate an amplification product. Amplification typically requires the presence of deoxyribonucleoside triphosphates, a DNA polymerase enzyme (e.g., Platinum® Taq) and an appropriate buffer and/or co-factors for optimal activity of the polymerase enzyme (e.g., MgCl₂ and/or KCl).

The term “primer” is used herein as known to those skilled in the art and refers to oligomeric compounds, primarily to oligonucleotides but also to modified oligonucleotides that are able to “prime” DNA synthesis by a template-dependent DNA polymerase, i.e., the 3′-end of the, e.g., oligonucleotide provides a free 3′—OH group in which further “nucleotides” may be attached by a template-dependent DNA polymerase establishing 3′ to 5′ phosphodiester linkage whereby deoxynucleoside triphosphates are used and whereby pyrophosphate is released. Therefore, there is—except possibly for the intended function—no fundamental difference between a “primer”, an “oligonucleotide”, an “oligonucleotide primer”, a “probe” or an “oligonucleotide probe”.

The term “hybridizing” refers to the annealing of one or more probes to an amplification product. Hybridization conditions typically include a temperature that is below the melting temperature of the probes but that avoids non-specific hybridization of the probes.

The term “5′ to 3′ nuclease activity” refers to an activity of a nucleic acid polymerase, typically associated with the nucleic acid strand synthesis, whereby nucleotides are removed from the 5′ end of nucleic acid strand.

The term “thermostable polymerase” refers to a polymerase enzyme that is heat stable, i.e., the enzyme catalyzes the formation of primer extension products complementary to a template and does not irreversibly denature when subjected to the elevated temperatures for the time necessary to effect denaturation of double-stranded template nucleic acids. Generally, the synthesis is initiated at the 3′ end of each primer and proceeds in the 5′ to 3′ direction along the template strand. Thermostable polymerases have been isolated from Thermus flavus, T. ruber, T. thermophilus, T. aquaticus, T. lacteus, T rubens, Bacillus stearothermophilus, and Methanothermus fervidus. Nonetheless, polymerases that are not thermostable also can be employed in PCR assays provided the enzyme is replenished.

The term “complement thereof” refers to nucleic acid that is both the same length as, and exactly complementary to, a given nucleic acid.

The term “extension” or “elongation” when used with respect to nucleic acids refers to when additional nucleotides (or other analogous molecules) are incorporated into the nucleic acids. For example, a nucleic acid is optionally extended by a nucleotide incorporating biocatalyst, such as a polymerase that typically adds nucleotides at the 3′ terminal end of a nucleic acid.

The terms “identical” or percent “identity” in the context of two or more nucleic acid sequences, refer to two or more sequences or subsequences that are the same or have a specified percentage of nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated herein by reference.

A “modified nucleotide” in the context of an oligonucleotide refers to an alteration in which at least one nucleotide of the oligonucleotide sequence is replaced by a different nucleotide that provides a desired property to the oligonucleotide. Exemplary modified nucleotides that can be substituted in the oligonucleotides described herein include, e.g., a C5-methyl-dC, a C5-ethyl-dC, a C5-methyl-dU, a C5-ethyl-dU, a 2,6-diaminopurine, a C5-propynyl-dC, a C5-propynyl-dU, a C7-propynyl-dA, a C7-propynyl-dG, a C5-propargylamino-dC, a C5-propargylamino-dU, a C7-propargylamino-dA, a C7-propargylamino-dG, a 7-deaza-2-deoxyxanthosine, a pyrazolo-pyrimidine analog, a pseudo-dU, a nitro pyrrole, a nitro indole, 2′-O-methyl Ribo-U, 2′-O-methyl Ribo-C, an N4-ethyl-dC, an N6-methyl-dA, and the like. Many other modified nucleotides that can be substituted in the oligonucleotides are referred to herein or are otherwise known in the art. In certain embodiments, modified nucleotide substitutions modify melting temperatures (Tm) of the oligonucleotides relative to the melting temperatures of corresponding unmodified oligonucleotides. To further illustrate, certain modified nucleotide substitutions can reduce non-specific nucleic acid amplification (e.g., minimize primer dimer formation or the like), increase the yield of an intended target amplicon, and/or the like in some embodiments. Examples of these types of nucleic acid modifications are described in, e.g., U.S. Pat. No. 6,001,611, which is incorporated herein by reference.

A “variant” of a given oligonucleotide may contain one or more nucleotide additions, deletions or substitutions such as one or more nucleotide additions, deletions or substitutions at the 5′ end and/or the 3′ end of the respective sequence of the oligonucleotide. As detailed above, a primer (and/or probe) may be chemically modified, i.e., a primer and/or probe may comprise a modified nucleotide or a non-nucleotide compound. A probe (or a primer) is then a modified oligonucleotide. “Modified nucleotides” (or “nucleotide analogs”) differ from a natural “nucleotide” by some modification but still consist of a base or base-like compound, a pentofuranosyl sugar or a pentofuranosyl sugar-like compound, a phosphate portion or phosphate-like portion, or combinations thereof. For example, a “label” may be attached to the base portion of a “nucleotide” whereby a “modified nucleotide” is obtained. A natural base in a “nucleotide” may also be replaced by, e.g., a 7-desazapurine whereby a “modified nucleotide” is obtained as well. The terms “modified nucleotide” or “nucleotide analog” are used interchangeably in the present application. A “modified nucleoside” (or “nucleoside analog”) differs from a natural nucleoside by some modification in the manner as outlined above for a “modified nucleotide” (or a “nucleotide analog”).

Oligonucleotides including modified oligonucleotides and oligonucleotide analogs that amplify a nucleic acid molecule for example, a nucleic acid molecule encoding the GBS 23S rRNA gene, the GBS cfb gene, or the clindamycin resistance gene (e.g., ermTR or lsaC) nucleic acid sequences, can be designed using, for example, a computer program such as OLIGO (Molecular Biology Insights Inc., Cascade, Colo.). Important features when designing oligonucleotides to be used as amplification primers include, but are not limited to, an appropriate size amplification product to facilitate detection (e.g., by electrophoresis), similar melting temperatures for the members of a pair of primers, and the length of each primer (i.e., the primers need to be long enough to anneal with sequence-specificity and to initiate synthesis but not so long that fidelity is reduced during oligonucleotide synthesis). Typically, oligonucleotide primers are 8 to 50 nucleotides in length (e.g., 8, 10, 12, 14, 16, 18, 20, 22, 24, 26, 28, 30, 32, 34, 36, 38, 40, 42, 44, 46, 48, or 50 nucleotides in length).

In addition to a set of primers, the methods may use one or more probes in order to detect the presence or absence of the GBS and the clindamycin-resistance mechanisms. The term “probe” refers to synthetically or biologically produced nucleic acids (DNA or RNA), which by design or selection, contain specific nucleotide sequences that allow them to hybridize under defined predetermined stringencies specifically (i.e., preferentially) to “target nucleic acids”, in the present case to an GBS 23S rRNA or GBS cfb (GBS target) nucleic acid and/or to a ermTR, ermB, ermT, lsaC, lsaE (clindamycin resistance target) nucleic acid. A “probe” can be referred to as a “detection probe” meaning that it detects the target nucleic acid.

In some embodiments, the described probes can be labeled with at least one fluorescent label. In one embodiment, the probes can be labeled with a donor fluorescent moiety, e.g., a fluorescent dye, and a corresponding acceptor fluorescent moiety, e.g., a quencher.

Designing oligonucleotides to be used as probes can be performed in a manner similar to the design of primers. Embodiments may use a single probe or a pair of probes for detection of the amplification product. Depending on the embodiment, the probe(s) use may comprise at least one label and/or at least one quencher moiety. As with the primers, the probes usually have similar melting temperatures, and the length of each probe must be sufficient for sequence-specific hybridization to occur but not so long that fidelity is reduced during synthesis. Oligonucleotide probes are generally 15 to 30 (e.g., 16, 18, 20, 21, 22, 23, 24, or 25) nucleotides in length.

Constructs can include vectors each containing one of the primers and probes nucleic acid molecules (e.g., SEQ ID NOs: 1-21). Constructs can be used, for example, as control template nucleic acid molecules. Vectors suitable for use are commercially available and/or produced by recombinant nucleic acid technology methods routine in the art. Target nucleic acid molecules can be obtained, for example, by chemical synthesis, direct cloning genes, or by PCR amplification. Constructs suitable for use in the methods typically include, in addition to target nucleic acid molecules (e.g., a nucleic acid molecule that contains one or more sequences of SEQ ID NOs: 1-21), sequences encoding a selectable marker (e.g., an antibiotic resistance gene) for selecting desired constructs and/or transformants, and an origin of replication. The choice of vector systems usually depends upon several factors, including, but not limited to, the choice of host cells, replication efficiency, selectability, inducibility, and the ease of recovery.

Constructs containing the target nucleic acid molecules can be propagated in a host cell. As used herein, the term host cell is meant to include prokaryotes and eukaryotes such as yeast, plant and animal cells. Prokaryotic hosts may include E. coli, Salmonella typhimurium, Serratia marcescens, and Bacillus subtilis. Eukaryotic hosts include yeasts such as S. cerevisiae, S. pombe, Pichia pastoris, mammalian cells such as COS cells or Chinese hamster ovary (CHO) cells, insect cells, and plant cells such as Arabidopsis thaliana and Nicotiana tabacum. A construct can be introduced into a host cell using any of the techniques commonly known to those of ordinary skill in the art. For example, calcium phosphate precipitation, electroporation, heat shock, lipofection, microinjection, and viral-mediated nucleic acid transfer are common methods for introducing nucleic acids into host cells. In addition, naked DNA can be delivered directly to cells (see, e.g., U.S. Pat. Nos. 5,580,859 and 5,589,466).

The term, “enriched sample” or “enriched specimen” means a sample or specimen that has been processed to increase the quantity or concentration of a target of interest that is suspected of being present in the sample. Specimens acquired from a patient may be enriched for one or more microorganisms present in the sample, such as GBS and other strains of Streptococcus. A number of culture-based and molecular methods exist for the enrichment of Streptococcal species, including GBS and other microorganisms in a specimen. In one example, a swab used to collect a specimen from a patient is placed into an elution medium (e.g., Liquid Amies). Thereafter, LIM enrichment broth (see, e.g., Lim, D. V., et al. 1982. Current Microbiol.; 7:99-101) is inoculated with the elution medium. The enrichment broth is then incubated for a period of time sufficient to selectively enrich for Streptococcal species, including GBS (e.g., 18-24 hours). The resulting enriched specimen may then be used for detection of GBS, one or more clindamycin resistance markers, and/or other targets of interest that may be present in the enriched sample. Patient specimens, including vaginal and rectal specimens, can be collected using commercially available swabs, such as ESWAB (COPAN).

The term, “direct from specimen” means a sample that is processed directly following collection without further enrichment, in contrast with an enriched sample. Direct from specimen samples include vaginal and rectal samples acquired with a swab and optionally placed into an elution medium. The swab and/or the elution medium may be direct tested without enrichment of microorganisms that may be present in the elution medium or on the swab.

### Polymerase Chain Reaction (PCR)

U.S. Pat. Nos. 4,683,202, 4,683,195, 4,800,159, and 4,965,188 disclose conventional PCR techniques. PCR typically employs two oligonucleotide primers that bind to a selected nucleic acid template (e.g., DNA or RNA). Primers useful in some embodiments include oligonucleotides capable of acting as points of initiation of nucleic acid synthesis within the described target gene and target allele nucleic acid sequences (e.g., SEQ ID NOs: 1, 2, 4, 5, 7, 8, 10, 11, and 13, 14). A primer can be purified from a restriction digest by conventional methods, or it can be produced synthetically. The primer is preferably single-stranded for maximum efficiency in amplification, but the primer can be double-stranded. Double-stranded primers are first denatured, i.e., treated to separate the strands. One method of denaturing double stranded nucleic acids is by heating.

If the template nucleic acid is double-stranded, it is necessary to separate the two strands before it can be used as a template in PCR. Strand separation can be accomplished by any suitable denaturing method including physical, chemical or enzymatic means. One method of separating the nucleic acid strands involves heating the nucleic acid until it is predominately denatured (e.g., greater than 50%, 60%, 70%, 80%, 90% or 95% denatured). The heating conditions necessary for denaturing template nucleic acid will depend, e.g., on the buffer salt concentration and the length and nucleotide composition of the nucleic acids being denatured, but typically range from about 90° C. to about 105° C. for a time depending on features of the reaction such as temperature and the nucleic acid length. Denaturation is typically performed for about 30 sec to 4 min (e.g., 1 min to 2 min 30 sec, or 1.5 min).

If the double-stranded template nucleic acid is denatured by heat, the reaction mixture is allowed to cool to a temperature that promotes annealing of each primer to its target sequence on the described nucleic acid molecules. The temperature for annealing is usually from about 35° C. to about 65° C. (e.g., about 40° C. to about 60° C.; about 45° C. to about 50° C.). Annealing times can be from about 10 sec to about 1 min (e.g., about 20 sec to about 50 sec; about 30 sec to about 40 sec). The reaction mixture is then adjusted to a temperature at which the activity of the polymerase is promoted or optimized, i.e., a temperature sufficient for extension to occur from the annealed primer to generate products complementary to the template nucleic acid. The temperature should be sufficient to synthesize an extension product from each primer that is annealed to a nucleic acid template, but should not be so high as to denature an extension product from its complementary template (e.g., the temperature for extension generally ranges from about 40° C. to about 80° C. (e.g., about 50° C. to about 70° C.; about 60° C.). Extension times can be from about 10 sec to about 5 min (e.g., about 30 sec to about 4 min; about 1 min to about 3 min; about 1 min 30 sec to about 2 min). PCR assays can employ the target gene and/or allele nucleic acid such as RNA or DNA (cDNA). The template nucleic acid need not be purified; it may be a minor fraction of a complex mixture, such as the target nucleic acid contained in biological samples. Target nucleic acid molecules may be extracted from a biological sample by routine techniques such as those described in Diagnostic Molecular Microbiology: Principles and Applications (Persing et al. (eds), 1993, American Society for Microbiology, Washington D.C.). Nucleic acids can be obtained from any number of sources, such as plasmids, or natural sources including bacteria, yeast, viruses, organelles, or higher organisms such as plants or animals.

The oligonucleotide primers are combined with PCR reagents under reaction conditions that induce primer extension. For example, chain extension reactions generally include 50 mM KCl, 10 mM Tris-HCl (pH 8.3), 15 mM MgCl₂, 0.001% (w/v) gelatin, 0.5-1.0 μg denatured template DNA, 50 pmoles of each oligonucleotide primer, 2.5 U of Taq polymerase, and 10% DMSO). The reactions usually contain 150 to 320 μM each of dATP, dCTP, dTTP, dGTP, or one or more analogs thereof.

The newly synthesized strands form a double-stranded molecule that can be used in the succeeding steps of the reaction. The steps of strand separation, annealing, and elongation can be repeated as often as needed to produce the desired quantity of amplification products corresponding to the target nucleic acid molecules. The limiting factors in the reaction are the amounts of primers, thermostable enzyme, and nucleoside triphosphates present in the reaction. The cycling steps (i.e., denaturation, annealing, and extension) are preferably repeated at least once. For use in detection, the number of cycling steps will depend, e.g., on the nature of the sample. If the sample is a complex mixture of nucleic acids, more cycling steps will be required to amplify the target sequence sufficient for detection. Generally, the cycling steps are repeated at least about 20 times, but may be repeated as many as 40, 60, or even 100 times.

### Fluorescence Resonance Energy Transfer (FRET)

FRET technology (see, for example, U.S. Pat. Nos. 4,996,143, 5,565,322, 5,849,489, and 6,162,603) is based on a concept that when a donor fluorescent moiety and a corresponding acceptor fluorescent moiety are positioned within a certain distance of each other, energy transfer takes place between the two fluorescent moieties that can be visualized or otherwise detected and/or quantitated. The donor typically transfers the energy to the acceptor when the donor is excited by light radiation with a suitable wavelength. The acceptor typically re-emits the transferred energy in the form of light radiation with a different wavelength. In certain systems, non-fluorescent energy can be transferred between donor and acceptor moieties, by way of biomolecules that include substantially non-fluorescent donor moieties (see, for example, U.S. Pat. No. 7,741,467).

In one example, a oligonucleotide probe can contain a donor fluorescent moiety and a corresponding quencher, which may or not be fluorescent, and which dissipates the transferred energy in a form other than light. When the probe is intact, energy transfer typically occurs between the two fluorescent moieties such that fluorescent emission from the donor fluorescent moiety is quenched. During an extension step of a polymerase chain reaction, a probe bound to an amplification product is cleaved by the 5′ to 3′ nuclease activity of, e.g., a Taq Polymerase such that the fluorescent emission of the donor fluorescent moiety is no longer quenched. Exemplary probes for this purpose are described in, e.g., U.S. Pat. Nos. 5,210,015, 5,994,056, and 6,171,785. Commonly used donor-acceptor pairs include the FAM-TAMRA pair. Commonly used quenchers are DABCYL and TAMRA. Commonly used dark quenchers include BlackHole Quenchers™ (BHQ), (Biosearch Technologies, Inc., Novato, Cal.), Iowa Black™, (Integrated DNA Tech., Inc., Coralville, Iowa), BlackBerry™ Quencher 650 (BBQ-650), (Berry & Assoc., Dexter, Mich.).

In another example, two oligonucleotide probes, each containing a fluorescent moiety, can hybridize to an amplification product at particular positions determined by the complementarity of the oligonucleotide probes to the target nucleic acid sequence. Upon hybridization of the oligonucleotide probes to the amplification product nucleic acid at the appropriate positions, a FRET signal is generated. Hybridization temperatures can range from about 35° C. to about 65° C. for about 10 sec to about 1 min.

Fluorescent analysis can be carried out using, for example, a photon counting epifluorescent microscope system (containing the appropriate dichroic mirror and filters for monitoring fluorescent emission at the particular range), a photon counting photomultiplier system, or a fluorimeter. Excitation to initiate energy transfer, or to allow direct detection of a fluorophore, can be carried out with an argon ion laser, a high intensity mercury (Hg) arc lamp, a fiber optic light source, or other high intensity light source appropriately filtered for excitation in the desired range. As used herein with respect to donor and corresponding acceptor fluorescent moieties “corresponding” refers to an acceptor fluorescent moiety having an absorbance spectrum that overlaps the emission spectrum of the donor fluorescent moiety. The wavelength maximum of the emission spectrum of the acceptor fluorescent moiety should be at least 100 nm greater than the wavelength maximum of the excitation spectrum of the donor fluorescent moiety. Accordingly, efficient non-radiative energy transfer can be produced therebetween.

Fluorescent donor and corresponding acceptor moieties are generally chosen for (a) high efficiency Forster energy transfer; (b) a large final Stokes shift (>100 nm); (c) shift of the emission as far as possible into the red portion of the visible spectrum (>600 nm); and (d) shift of the emission to a higher wavelength than the Raman water fluorescent emission produced by excitation at the donor excitation wavelength. For example, a donor fluorescent moiety can be chosen that has its excitation maximum near a laser line (for example, Helium-Cadmium 442 nm or Argon 488 nm), a high extinction coefficient, a high quantum yield, and a good overlap of its fluorescent emission with the excitation spectrum of the corresponding acceptor fluorescent moiety. A corresponding acceptor fluorescent moiety can be chosen that has a high extinction coefficient, a high quantum yield, a good overlap of its excitation with the emission of the donor fluorescent moiety, and emission in the red part of the visible spectrum (>600 nm).

Representative donor fluorescent moieties that can be used with various acceptor fluorescent moieties in FRET technology include fluorescein, Lucifer Yellow, B-phycoerythrin, 9-acridineisothiocyanate, Lucifer Yellow VS, 4-acetamido-4′-isothio-cyanatostilbene-2,2′-disulfonic acid, 7-diethylamino-3-(4′-isothiocyanatophenyl)-4-methylcoumarin, succinimdyl 1-pyrenebutyrate, and 4-acetamido-4′-isothiocyanatostilbene-2,2′-disulfonic acid derivatives. Representative acceptor fluorescent moieties, depending upon the donor fluorescent moiety used, include LC Red 640, LC Red 705, Cy5, Cy5.5, Lissamine rhodamine B sulfonyl chloride, tetramethyl rhodamine isothiocyanate, rhodamine x isothiocyanate, erythrosine isothiocyanate, fluorescein, diethylenetriamine pentaacetate, or other chelates of Lanthanide ions (e.g., Europium, or Terbium). Donor and acceptor fluorescent moieties can be obtained, for example, from Molecular Probes (Junction City, OR) or Sigma Chemical Co. (St. Louis, MO).

The donor and acceptor fluorescent moieties can be attached to the appropriate probe oligonucleotide via a linker arm. The length of each linker arm is important, as the linker arms will affect the distance between the donor and acceptor fluorescent moieties. The length of a linker arm can be the distance in Angstroms (Å) from the nucleotide base to the fluorescent moiety. In general, a linker arm is from about 10 Å to about 25 Å. The linker arm may be of the kind described in WO 84/03285. WO 84/03285 also discloses methods for attaching linker arms to a particular nucleotide base, and also for attaching fluorescent moieties to a linker arm.

An acceptor fluorescent moiety, such as an LC Red 640, can be combined with an oligonucleotide which contains an amino linker (e.g., C6-amino phosphoramidites available from ABI (Foster City, CA) or Glen Research (Sterling, VA)) to produce, for example, LC Red 640-labeled oligonucleotide. Frequently used linkers to couple a donor fluorescent moiety such as fluorescein to an oligonucleotide include thiourea linkers (FITC-derived, for example, fluorescein-CPG's from Glen Research or ChemGene (Ashland, MA)), amide-linkers (fluorescein-NHS-ester-derived, such as CX-fluorescein-CPG from BioGenex (San Ramon, CA)), or 3′-amino-CPGs that require coupling of a fluorescein-NHS-ester after oligonucleotide synthesis

### Detection of Target GBS Genes and Clindamycin-Resistance Genes

The present disclosure provides methods for detecting the presence or absence of the GBS 23S rRNA gene, the GBS cfb gene, and the ermTR, ermB, ermT lsaC and lsaE genes in a biological or non-biological sample. Methods provided avoid problems of sample contamination, false negatives, and false positives. The methods include performing at least one cycling step that includes amplifying a portion of the target nucleic acid molecules from a sample using a plurality of pairs of target primers, and a FRET detecting step. Multiple cycling steps are performed, preferably in a thermocycler. Methods can be performed using the target primers and probes to detect the presence of the target gene, and the detection of the amplification product in the assay indicates the presence of the target gene and/or target allele in the sample.

As described herein, amplification products can be detected using labeled hybridization probes that take advantage of FRET technology. One FRET format utilizes TaqMan® technology to detect the presence or absence of an amplification product, and hence, the presence or absence of GBS and/or clindamycin resistance genes. TaqMan® technology utilizes one single-stranded hybridization probe labeled with, e.g., one fluorescent dye and one quencher, which may or may not be fluorescent. When a first fluorescent moiety is excited with light of a suitable wavelength, the absorbed energy is transferred to a second fluorescent moiety according to the principles of FRET. The second fluorescent moiety is generally a quencher molecule. During the annealing step of the PCR reaction, the labeled hybridization probe binds to the target DNA (i.e., the amplification product) and is degraded by the 5′ to 3′ nuclease activity of, e.g., the Taq Polymerase during the subsequent elongation phase. As a result, the fluorescent moiety and the quencher moiety become spatially separated from one another. As a consequence, upon excitation of the first fluorescent moiety in the absence of the quencher, the fluorescence emission from the first fluorescent moiety can be detected. By way of example, an ABI PRISM® 7700 Sequence Detection System (Applied Biosystems) uses TaqMan® technology, and is suitable for performing the methods described herein for detecting the presence or absence of GBS and/or clindamycin resistance genes in the sample.

Molecular beacons in conjunction with FRET can also be used to detect the presence of an amplification product using the real-time PCR methods. Molecular beacon technology uses a hybridization probe labeled with a first fluorescent moiety and a second fluorescent moiety. The second fluorescent moiety is generally a quencher, and the fluorescent labels are typically located at each end of the probe. Molecular beacon technology uses a probe oligonucleotide having sequences that permit secondary structure formation (e.g., a hairpin). As a result of secondary structure formation within the probe, both fluorescent moieties are in spatial proximity when the probe is in solution. After hybridization to the target nucleic acids (i.e., amplification products), the secondary structure of the probe is disrupted and the fluorescent moieties become separated from one another such that after excitation with light of a suitable wavelength, the emission of the first fluorescent moiety can be detected.

Another common format of FRET technology utilizes two hybridization probes. Each probe can be labeled with a different fluorescent moiety and are generally designed to hybridize in close proximity to each other in a target DNA molecule (e.g., an amplification product). A donor fluorescent moiety, for example, fluorescein, is excited at 470 nm by the light source of the LightCycler® Instrument. During FRET, the fluorescein transfers its energy to an acceptor fluorescent moiety such as LightCycler®-Red 640 (LC Red 640) or LightCycler®-Red 705 (LC Red 705). The acceptor fluorescent moiety then emits light of a longer wavelength, which is detected by the optical detection system of the LightCycler® instrument. Efficient FRET can only take place when the fluorescent moieties are in direct local proximity and when the emission spectrum of the donor fluorescent moiety overlaps with the absorption spectrum of the acceptor fluorescent moiety. The intensity of the emitted signal can be correlated with the number of original target DNA molecules. If amplification of target nucleic acid occurs and an amplification product is produced, the step of hybridizing results in a detectable signal based upon FRET between the members of the pair of probes.

Generally, the presence of FRET indicates the presence of the target sequence in the sample, and the absence of FRET indicates the absence of the target sequence in the sample. Inadequate specimen collection, transportation delays, inappropriate transportation conditions, or use of certain collection swabs (calcium alginate or aluminum shaft) are all conditions that can affect the success and/or accuracy of a test result, however. Using the methods disclosed herein, detection of FRET within, e.g., 45 cycling steps is indicative of a GBS infection.

Representative biological samples that can be used in practicing the methods include, but are not limited to dermal swabs, nasal swabs, wound swabs, blood cultures, skin, and soft tissue infections. Collection and storage methods of biological samples are known to those of skill in the art. Biological samples can be processed (e.g., by nucleic acid extraction methods and/or kits known in the art) to release target gene nucleic acid or in some cases, the biological sample can be contacted directly with the PCR reaction components and the appropriate oligonucleotides.

Melting curve analysis is an additional step that can be included in a cycling profile. Melting curve analysis is based on the fact that DNA melts at a characteristic temperature called the melting temperature (Tm), which is defined as the temperature at which half of the DNA duplexes have separated into single strands. The melting temperature of a DNA depends primarily upon its nucleotide composition. Thus, DNA molecules rich in G and C nucleotides have a higher Tm than those having an abundance of A and T nucleotides. By detecting the temperature at which signal is lost, the melting temperature of probes can be determined. Similarly, by detecting the temperature at which signal is generated, the annealing temperature of probes can be determined. The melting temperature(s) of the probes from the amplification products can confirm the presence or absence of the target sequence in the sample.

Within each thermocycler run, control samples can be cycled as well. Positive control samples can amplify target nucleic acid control template (other than described amplification products of target genes) using, for example, control primers and control probes. Positive control samples can also amplify, for example, a plasmid construct containing the target nucleic acid molecules. Such a plasmid control can be amplified internally (e.g., within the sample) or in a separate sample run side-by-side with the patients' samples using the same primers and probe as used for detection of the intended target. Such controls are indicators of the success or failure of the amplification, hybridization, and/or FRET reaction. Each thermocycler run can also include a negative control that, for example, lacks target template DNA. Negative control can measure contamination. This ensures that the system and reagents would not give rise to a false positive signal. Therefore, control reactions can readily determine, for example, the ability of primers to anneal with sequence-specificity and to initiate elongation, as well as the ability of probes to hybridize with sequence-specificity and for FRET to occur.

In an embodiment, the methods include steps to avoid contamination. For example, an enzymatic method utilizing uracil-DNA glycosylase is described in U.S. Pat. Nos. 5,035,996, 5,683,896 and 5,945,313 to reduce or eliminate contamination between one thermocycler run and the next. Conventional PCR methods in conjunction with FRET technology can be used to practice the methods. In one embodiment, a LightCycler® instrument is used. The following patent applications describe real-time PCR as used in the LightCycler® technology: WO 97/46707, WO 97/46714, and WO 97/46712.

The LightCycler® can be operated using a PC workstation and can utilize a Windows NT operating system. Signals from the samples are obtained as the machine positions the capillaries sequentially over the optical unit. The software can display the fluorescence signals in real-time immediately after each measurement. Fluorescent acquisition time is 10-100 milliseconds (msec). After each cycling step, a quantitative display of fluorescence vs. cycle number can be continually updated for all samples. The data generated can be stored for further analysis.

As an alternative to FRET, an amplification product can be detected using a double-stranded DNA binding dye such as a fluorescent DNA binding dye (e.g., SYBR® Green or SYBR® Gold (Molecular Probes)). Upon interaction with the double-stranded nucleic acid, such fluorescent DNA binding dyes emit a fluorescence signal after excitation with light at a suitable wavelength. A double-stranded DNA binding dye such as a nucleic acid intercalating dye also can be used.

When double-stranded DNA binding dyes are used, a melting curve analysis is usually performed for confirmation of the presence of the amplification product.

It is understood that the embodiments of the present disclosure are not limited by the configuration of one or more commercially available instruments.

### Articles of Manufacture/Kits

Embodiments of the present disclosure further provide for articles of manufacture or kits to detect the 23S rRNA and cfb genes of GBS, and the ermTR, ermB, ermT, lsaC and lsaE genes (i.e. genes responsible for clindamycin resistant GBS). An article of manufacture can include primers and probes used to detect clindamycin resistant GBS, together with suitable packaging materials. Representative primers and probes for detection of clindamycin resistant GBS are capable of hybridizing to target nucleic acid molecules. In addition, the kits may also include suitably packaged reagents and materials needed for DNA immobilization, hybridization, and detection, such solid supports, buffers, enzymes, and DNA standards. Methods of designing primers and probes are disclosed herein, and representative examples of primers and probes that amplify and hybridize to target nucleic acid molecules are provided.

Articles of manufacture can also include one or more fluorescent moieties for labeling the probes or, alternatively, the probes supplied with the kit can be labeled. For example, an article of manufacture may include a donor and/or an acceptor fluorescent moiety for labeling the probes. Examples of suitable FRET donor fluorescent moieties and corresponding acceptor fluorescent moieties are provided above.

Articles of manufacture can also contain a package insert or package label having instructions thereon for using the target primers and probes to detect clindamycin resistant GBS in a sample.

Articles of manufacture may additionally include reagents for carrying out the methods disclosed herein (e.g., buffers, polymerase enzymes, co-factors, or agents to prevent contamination). Such reagents may be specific for one of the commercially available instruments described herein.

Embodiments of the present disclosure will be further described in the following examples, which do not limit the scope of the invention described in the claims.

### Examples

The following examples and figures are provided to aid the understanding of the subject matter, the true scope of which is set forth in the appended claims. It is understood that modifications can be made in the procedures set forth without departing from the spirit of the invention.

### Example 1: Primer and Probe Sequences

Table 1 shows the primers and probes that are used in the multiplex PCR assays for the detection of GBS and the clindamycin-resistance mechanisms according to the present disclosure.

TABLE 1

| SEQ ID | | | |
|---|---|---|---|
| NO | NAME | DESCRIPTION | SEQUENCE |
| 1 | SEGP3784 | GBS 23S rRNA | GACTGCGATATAGGATTAATCAT |
| | | forward primer | TATAGAAGJ |
| 2 | SEGP3785 | GBS 23S rRNA | GATGGTCCTCCCAGATTCCGJ |
| | | reverse primer | |
| 3 | SEGP4189 | GBS 23S rRNA probe | <HEX_Thr>ATGATAQGATTAACC |
| | | | TTAGCAGTATCCTGAGTACGGC< |
| | | | Spc_C3> |
| 4 | SEGP3786 | GBS cfb forward | TGCATTGTTATTTTCACCAGCTG |
| | | primer | TATTJ |
| 5 | GBS_cfb.R.4 | GBS cfb reverse | CCATTTGCTGGGCTTGATTATTA |
| | | primer | K |
| 6 | Cfb5_HEX_5 | GBS cfb probe | <HEX_Thr>TGCTGQATCAAGTGA |
| | | | CAACTCCACAAGTGGTAAATC |
| | | | <Spc_C3> |
| 7 | ermTR_F_15_46 | ermTR forward primer | CCCGAAAAATACGCAAAATTTC |
| | | | ATTACATCTJ |
| 8 | ermTR_R_204_175 | ermTR reverse primer | CGTGGAATGACATAAACCTTCAT |
| | | | CAATCTK |
| 9 | ermTR8.Fam | ermTR probe | <FAM_Thr>TTGGGTQCAGGAAAA |
| | | | GGACATTTTACCAAGGAACTTGT |
| | | | GGA<Spc_C3> |
| 10 | ermB_F_301_331 | ermB forward primer | TTCCTTACCATTTAAGCACACAA |
| | | | ATTATTAJ |
| 11 | ermB_R_472_452 | ermB reverse primer | GGCAGCTTAAGCAATTGCTGJ |
| 12 | ermB6.Fam | ermB probe | <FAM_Thr>TACCTTGQGATATTC |
| | | | ACCGAACACTAGGGTTGCTC |
| | | | <Spc_C3> |
| 13 | ermT6.F | ermT forward primer | TATAAAAGATAGTCAAAACTTT |
| | | | ATTACTTCAAAGK |
| 14 | ermT6.R | ermT reverse primer | GGATCTATTTCAATGGCGGTTAC |
| | | | ATAJ |
| 15 | ermT6.Fam | ermT probe | <FAM_Thr>TTGAGAQTTGGTTCA |
| | | | GGGAAAGGTCATTTCACGTT |
| | | | <Spc<SpcC3> |
| 16 | SEGP4021 | lsaC forward primer | AGAATTATTTGAAATGGATATG |
| | | | AAGAACTATAGK |
| 17 | SEGP4022 | IsaC reverse primer | TCTTTTATAATTTCCTCAATCTGT |
| | | | ATTCTTGATJ |
| 18 | SEGP4023 | lsaC probe | <JA270_Thr>AAAAAGAAAQGTTT |
| | | | TAATTGCTGTAAGTTTGTCAAAG |
| | | | GCAGCTCA<Spc_C3> |
| 19 | 186872 | lsaE forward primer | TCAAGAAGATGCTTTATATCGTC |
| | | | CGTTTAJ |
| 20 | 186873 | IsaE reverse primer | AGGTTCATCAATAAGCAGGAAA |
| | | | CAACTK |
| 21 | SEGP3912 | IsaE probe | <JA270_Thr>AATGGTGAGQCAAA |
| | | | CGAAGGTCCTTCTTGCAGCT |
| | | | <Spc_C3> |

Where indicated, the primer and probe sequences in Table 1 are illustrated as including modifications denoted according to the following scheme: J=t-butylbenzyl-dA, <HEX_Thr>=HEX dye, Q=BHQ2, <Spc_C3>=3′ spacer, K=t-butylbenzyl-dC, <FAM_Thr>=FAM dye, and <JA270_Thr>=JA270 dye.

### Example 2: PCR Experimental Conditions

Real-time PCR detection of the gene targets was performed using the LightCycler® 480 system (Roche Molecular Systems, Inc., Pleasanton, CA). The final concentrations of the amplification reagents and the thermoprofile used for PCR amplification reaction are show in Table 2:

TABLE 2

| | | |
|---|---|---|
| Reagent | Test Conc | 1 rxn (uL) |

Mg Reagent (R1)

| | | |
|---|---|---|
| Mn Reagent [R1] | 3.3 mM | 10.0 |
| Elution Buffer | — | 14.1 |
| Enzyme (Z05D) | — | 0.225 |
| IC | — | 5.0 |
| | Total | 29.4 |

Master Mix (R2) - Universal

| | | |
|---|---|---|
| Bulk Cobas 6800/8800 MMx-R2 | — | 12 |
| Buffer | | |
| Nuclease Free Water | — | 1.94 |
| UNG | — | 0.2 |
| NTQ21-46A - Aptamer | — | 0.056 |
| | Total | 14.2 |

PCR Thermocycle Parameters

| | | | Hold Time | |
|---|---|---|---|---|
| Step | Cycles | Temp (C.) | (hh:mm:ss) | Ramp |
| Pre-PCR | 1 | 50 | 0:02:00 | 4.4 |
| | | 94 | 0:00:05 | 4.4 |
| | | 55 | 0:02:00 | 2.2 |
| | | 60 | 0:06:00 | 4.4 |
| | | 65 | 0:04:00 | 4.4 |
| First Measurement | 5 | 95 | 0:00:05 | 4.4 |
| | | 55.0 | 0:00:30 | 2.2 |
| Second Measurement | 45 | 91.0 | 0:00:05 | 4.4 |
| | | 58.0 | 0:00:25 | 2.2 |
| Post | 1 | 40 | 0:02:00 | 2.2 |

The Pre-PCR program comprised initial denaturing and incubation at 55° C., 60° C. and 65° C. for reverse transcription of RNA templates. Incubating at three temperatures combines the advantageous effects that at lower temperatures slightly mismatched target sequences (such as genetic variants of an organism) are also transcribed, while at higher temperatures the formation of RNA secondary structures is suppressed, thus leading to a more efficient transcription. PCR cycling was divided into two measurements, wherein both measurements apply a one-step setup (combining annealing and extension). The first 5 cycles at 55° C. allow for an increased inclusivity by pre-amplifying slightly mismatched target sequences, whereas the 45 cycles of the second measurement provide for an increased specificity by using an annealing/extension temperature of 58° C.

### Example 3: Performance of PCR Assay for Detection of GBS and Clindamycin Resistance Genes

Results of PCR assay using the primers and probes for detecting GBS and ermTR in multiplex are shown in Table 3. The master mix used forward primers SEQ ID NO:1, SEQ ID NO:4, SEQ ID NO:7, the reverse primers SEQ ID NO:2, SEQ ID NO:5, SEQ ID NO:8 and probes SEQ ID NO:3, SEQ ID NO:6, SEQ ID NO:9. Template for GBS detection was genomic DNA extracted from a Streptococcus agalactiae clinical isolate at concentrations of 1.00E+06 copies per reaction to 1.00E+00 copies per reaction. Template plasmid carrying the ermTR gene at concentrations of 1.52E+05 copies per reaction to 1.53E+00 copies per reaction were tested. Initial testing demonstrated these primer/probe pairings produced detection in a multiplex assay.

Primer/probe pairings for ermB in singleplex and ermT in singleplex are shown in Table 4. The master mix used forward primer SEQ ID NO: 10, reverse primers SEQ ID NO: 11 and probe SEQ ID NO:12 for ermB. The master mix used forward primer SEQ ID NO: 13, reverse primers SEQ ID NO: 14 and probe SEQ ID NO: 15 for ermT. Primer/probe pairings for lsaC in singleplex and lsaE in singleplex are shown in Table 5. The master mix used forward primer SEQ ID NO: 16, reverse primers SEQ ID NO: 17 and probe SEQ ID NO: 18 for lsaC. The master mix used forward primer SEQ ID NO: 19, reverse primers SEQ ID NO: 20 and probe SEQ ID NO: 21 for lsaE. Template plasmid carrying the ermB, lsaC or lsaE gene were tested at concentrations of 1.00E+06 copies per reaction to 1.00E+00 copies per reaction. Template plasmid carrying the ermT gene were tested at concentrations of 7.95E+03 copies per reaction to 7.95E+00 copier per reaction.

TABLE 3
Mastermix

| | | |
|---|---|---|
| Test Conc. (cp/rxn) | Avg Ct. | Avg RFI |

GBS detection

| | | |
|---|---|---|
| 1.00E+06 | 14.0 | 31.6 |
| 1.00E+05 | 17.4 | 30.9 |
| 1.00E+04 | 21.0 | 30.4 |
| 1.00E+03 | 24.6 | 30.0 |
| 1.00E+02 | 28.1 | 29.3 |
| 1.00E+01 | 31.5 | 25.3 |
| 1.00E+00 | 34.3 | 19.7 |
| NTC | . | . |

ermTR detection

| | | |
|---|---|---|
| — | — | — |
| 1.53E+05 | 23.1 | 18.9 |
| 1.53E+04 | 28.4 | 18.5 |
| 1.53E+03 | 32.9 | 17.7 |
| 1.53E+02 | 36.2 | 14.3 |
| 1.53E+01 | 39.1 | 4.5 |
| 1.53E+00 | 43.3 | 4.2 |
| NTC | . | . |

TABLE 4
Mastermix

| | | |
|---|---|---|
| Test Conc. (cp/rxn) | Avg Ct. | Avg RFI |

ermB detection

| | | |
|---|---|---|
| 1.00E+06 | 16.4 | 23.8 |
| 1.00E+05 | 20.3 | 23.2 |
| 1.00E+04 | 24.0 | 22.7 |
| 1.00E+03 | 27.1 | 22.2 |
| 1.00E+02 | 31.1 | 20.5 |
| 1.00E+01 | 35.0 | 15.2 |
| 1.00E+00 | 37.0 | 5.9 |
| NTC | . | 1.0 |

ermT detection

| | | |
|---|---|---|
| 7.95E+03 | 24.7 | 20.7 |
| 7.95E+02 | 29.2 | 19.5 |
| 7.95E+01 | 35.6 | 11.7 |
| 7.95E+00 | 37.0 | 10.4 |
| NTC | . | . |

TABLE 5
Mastermix

| | | |
|---|---|---|
| Test Conc. (cp/rxn) | Avg Ct. | Avg RFI |

lsaC detection

| | | |
|---|---|---|
| 1.00E+06 | 23.7 | 5.1 |
| 1.00E+05 | 27.2 | 5.1 |
| 1.00E+04 | 31.2 | 5.0 |
| 1.00E+03 | 33.8 | 4.8 |
| 1.00E+02 | 38.6 | 2.7 |
| 1.00E+01 | 41.2 | 1.3 |
| 1.00E+00 | 29.4 | 2.3 |
| NTC | . | . |

lsaE detection

| | | |
|---|---|---|
| 1.00E+06 | 18.5 | 9.1 |
| 1.00E+05 | 22.1 | 9.1 |
| 1.00E+04 | 25.6 | 9.0 |
| 1.00E+03 | 28.9 | 8.8 |
| 1.00E+02 | 32.2 | 7.6 |
| 1.00E+01 | 33.8 | 5.6 |
| 1.00E+00 | 34.4 | 4.6 |
| NTC | . | . |

In Tables 3-5, Test Conc. (cp/rxn) is the test concentration in units of copies per reaction, Avg Ct. is the average cycle threshold (i.e., the average number of cycles required for the fluorescence signal to cross a background fluorescence signal threshold), and Avg RFI is the average relative fluorescence intensity.

### Example 4: Further Primer and Probe Sequences

Table 6 shows further primers and probes that are used in the multiplex PCR assays for the detection of GBS and the clindamycin-resistance mechanisms according to the present disclosure.

TABLE 6

| SEQ | | | |
|---|---|---|---|
| ID NO | NAME | DESCRIPTION | SEQUENCE |
| 22 | GBS021 | GBS 23S rRNA | GGGGTTGTAGGACTGCGATATAG |
| | | forward primer | GAST |
| 23 | GBS071 | GBS 23S rRNA | GLGGTTGTAGGACTGCGATATAG |
| | | forward primer | GAMT |
| 24 | GBS025 | GBS 23S rRNA | CGTACTCAGGATACTGCTAAGGT |
| | | reverse primer | TAATCTATCJ |
| 25 | GBS051 | GBS 23S rRNA | <HEX_Thr>TNATCQATTATAGAA |
| | | probe HEXQ5 | GAATTACCTGGGAAGGTAAGCCA |
| | | | AAGAGAGTAAC <Spc_C3> |
| 26 | GBS027 | GBS 23SrRNA | <FAM_Thr>TAATCAQTTATAGAA |
| | | probe Fam | GAATTACCTGGGAAGGTAAGCCA |
| | | | AAGAGAGTAAC<Spc_C3> |
| 27 | GBS069B | GBS 23S S. urinalis | <HEX_Thr>AATOATQTATAGAAG |
| | | exclusivity probe | AATTACCTGGGAAGGTAAGCCAA |
| | | | AGAGAGTAAC <Spc_C3> |
| 28 | GBS031 | cfb forward primer | TGCATTGTTATTTTCACCAGCTGT |
| | | | ATTAGAJ |
| 29 | GBS033 | cfb reverse primer | TTTTGAGCCATTTGCTGGGCTTG |
| | | | ATTATTJ |
| 30 | GBS004 | cfb probe | <HEX_Thr>TPCTGAQTCAAGTGAC |
| | | | AACTCCACAAGTGGTAAATC |
| | | | <Spc_C3> |
| 31 | GBS039 | cfb inclusive | ATTGTTTTCTCCAGCGATTTTGGA |
| | | forward primer | AGTTK |
| 32 | GBS041 | cfb inclusive reverse | TCAAGTTTTTGTTTTATTTGTTGT |
| | | primer | TGATTATTAAK |
| 64 | GBS059 | cfb inclusive probe | <HEX_Thr>TPCTGQATCAAGCTAC |
| | | | TAATCATGTCGTAGTTAGTC |
| | | | <Spc_C3> |
| 33 | ermB4.F | ermB forward | AAAATTGTTGGGAATATTCCTTA |
| | | primer | CCATTTAJ |
| 34 | ermB_R_462_440 | ermB reverse primer | GCAATTGCTGAATCGAGACTTGJ |
| 35, 36 | GBS006 | ermB probe | <JA270_Thr>TNCCTTGGATAQTTC |
| | | | ACCGAACACTAGGGTTGCTC |
| | | | <Spc_C3> |
| 13 | ermT6.F | ermT forward primer | TATAAAAGATAGTCAAAACTTTA |
| | | | TTACTTCAAAGK |
| 14 | ermT6.R | ermT reverse primer | GGATCTATTTCAATGGCGGTTAC |
| | | | ATAJ |
| 37 | GBS010 | ermT probe | <FAM_Thr>TRGAGAQTTGGTTCA |
| | | | GGGAAAGGTCATTTCACGTT |
| | | | <Spc_C3> |
| 38 | ermTR_F_35_69T | ermTR forward | TCATTACATCTAAAAAGCATGTA |
| | BB | primer | AAGGAAATATTJ |
| 39 | ermTR_R_203_171 | ermTR reverse | GTGGAATGACATAAACCTTCATC |
| | tBB | primer | AATCTCTATJ |
| 40, 41 | ermTR8.JA270_10 | ermTR probe | <JA270_Thr>TTGGGTCAGGQAAA |
| | | | AGGACATTTTACCAAGGAACTTG |
| | | | TGGA <Spc_C3> |
| 42 | SEG4091 | IsaC forward primer | GGTGATGTAAAATTAGCAAGTAA |
| | | | TTTAAAAATATK |
| 43 | SEG4092 | IsaC reverse primer | TCGCTATAGTTCTTCATATCCATT |
| | | | TCAAATJ |
| 44 | GBS012 | IsaC probe | <FAM_Thr>APCAAGQGTGTTGAT |
| | | | GAAACATTGTGCAAAACAATTCT |
| | | | TAGAA <Spc_C3> |
| 19 | SEGP3910 | IsaE forward primer | TCAAGAAGATGCTTTATATCGTC |
| | | | CGTTTAJ |
| 20 | SEGP3911 | IsaE reverse primer | AGGTTCATCAATAAGCAGGAAAC |
| | | | AACTK |
| 45 | GBS015 | IsaE probe | <FAM_Thr>ANTGGTQGAGCAAAC |
| | | | GAAGGTCCTTCTTGCAGCT |
| | | | <Spc_C3> |
| 46 | GBS084 | GBS 23S probe to | <HEX_Thr>AARVNTTQATAGAAG |
| | | improve specificity | AATTACCTGGGAAGGTAAGC |
| | | | <Spc_C3> |
| 47 | GBS085 | S. urinalis 23S | AARPNTTQATAGAAGAATTACCT |
| | | blocking probe | GGGAAGGTAAGC<Spc_C3> |
| 48 | GBS086 | GBS 23S probe to | <HEX_Thr>AARVNTTAQTAGAAG |
| | | improve specificity | AATTACCTGGGAAGGTAAGC |
| | | | <SpcC3> |
| 49 | GBS087 | S. urinalis 23S | AARPNTTAQTAGAAGAATTACCT |
| | | blocking probe | GGGAAGGTAAGC<Spc_C3> |
| 50 | GBS088B | GBS 23S probe to | <HEX_Thr>AARVNQTTATAGAAG |
| | | improve specificity | AATTACCTGGGAAGGTAAGC |
| | | | <Spc_C3> |
| 51 | GBS089 | S. urinalis 23S | AARPNQTTATAGAAGAATTACCT |
| | | blocking probe | GGGAAGGTAAGC<Spc_C3> |
| 52 | GBS090 | GBS 23S probe to | <HEX_Thr>TAARVNTTQATAGAA |
| | | improve specificity | GAATTACCTGGGAAGGTAAGC |
| | | | <Spc_C3> |
| 53 | GBS091 | S. urinalis 23S | TAARPNTTQATAGAAGAATTACC |
| | | blocking probe | TGGGAAGGTAAGC<Spc_C3> |
| 54 | GBS092 | GBS 23S probe to | <HEX_Thr>TAARVNTTAQTAGAA |
| | | improve specificity | GAATTACCTGGGAAGGTAAGC |
| | | | <Spc_C3> |
| 55 | GBS093 | S. urinalis 23S | TAARPNTTAQTAGAAGAATTACC |
| | | blocking probe | TGGGAAGGTAAGC<Spc_C3> |
| 56 | GBS094 | GBS 23S probe to | <HEX_Thr>TAARVNQTTATAGAA |
| | | improve specificity | GAATTACCTGGGAAGGTAAGC |
| | | | <Spc_C3> |
| 57 | GBS095 | S. urinalis 23S | TAARPNQTTATAGAAGAATTACC |
| | | blocking probe | TGGGAAGGTAAGC<Spc_C3> |
| 58 | GBS096 | GBS 23S probe to | <HEEX_Thr>AARVRQTTATAGAAG |
| | | improve specificity | AATTACCTGGGAAGGTAAGCCAA |
| | | | AGAGAGTAAC<Spc_C3> |
| 59 | GBS097 | S. urinalis 23S | AARPRQTTATAGAAGAATTACCT |
| | | blocking probe | GGGAAGGTAAGCCAAAGAGAGT |
| | | | AAC<Spc_C3> |
| 60 | GBS080 | cfb Coumarin probe | <Coum_Thr>TGVTGATCQAAGTGA |
| | | | CAACTCCACAAGTGGTAAATC |
| | | | <Spc_C3> |
| 61 | GBS081 | cfb Coumarin probe | <Coum_Thr>TGVTGATCAQAGTGA |
| | | | CAACTCCACAAGTGGTAAATC |
| | | | <Spc_C3> |
| 62 | GBS082 | cfb Coumarin probe | <Coum_Thr>TGVTGATCQAAGCTA |
| | | | CTAATCATGTCGTAGTTAGTC |
| | | | <Spc_C3> |
| 63 | GBS083 | cfb Coumarin probe | <Coum_Thr>TGVTGATCAQAGCTA |
| | | | CTAATCATGTCGTAGTTAGTC |
| | | | <Spc_C3> |

Where indicated, the primer and probe sequences in Table 6 are illustrated as including modifications denoted according to the following scheme: J=t-butylbenzyl-dA, K=t-butylbenzyl-dC, L=7-deaza-dG, M=2′-O-Methyl-rU, N=D_LNA_A, O=N4-Ethyl-dC, P=D_LNA_G, R=D_LNA_T, S=2′-OMethyl-riboU, V=D_LNA_5-methyl-C, <Spc_C3>=3′ spacer, <Coum_Thr>=Coumarin dye, <FAM_Thr>=FAM dye, <HEX_Thr>=HEX dye, <JA270_Thr>=JA270 dye, and Q=BHQ2.

### Example 5: Performance of Assay for Detection of GBS

The current standard of care for the detection and identification of GBS is an enrichment-based approach characterized by a lengthy turn-around time of at least a full day. This approach generally involves collection of a specimen with a swab, release of the specimen from the swab in a liquid media, enrichment via culturing of the specimen in the liquid media for 18-24 hours, followed by either further culturing of the enriched specimen on agar plates or the use of a nucleic acid amplification test (NAAT).

The current understanding in the field is that the sensitivity of GBS detection is strongly impacted by culture enrichment. Accordingly, it is strongly recommended to incubate GBS screening specimens in selective enrichment broth irrespective of whether the ultimate detection method contemplates agar media plating or NAAT. This recommendation is based, at least in part, on reports that incubation in broth media prior to plating increases sensitivity of screening methods by about two-fold compared to direct from specimen plating. Likewise NAAT sensitivity is increased following culture enrichment. For example, the American Society for Microbiology Clinical and Public Health Microbiology Committee, Subcommittee on Laboratory Practices (ASM) recommends that “whether culture or NAAT are selected as the primary method of GBS detection, enrichment broth culture must first be performed.” The ASM guidelines go on to state, “Importantly, commercial NAATs performed directly from specimen (without enrichment) are available, but their use is not recommended at this time due to high false negative rates of 6.3% to 22%.”

While the ASM recommends against the use of existing commercial NAATs performed directly from specimen (without enrichment), it may be useful to provide for an approach that reduces or eliminates the need for an enrichment step in order to reduce the overall turnaround time for the detection and identification of GBS. For example, direct from specimen testing is useful for intrapartum testing where it is desirable to have results faster. Moreover, current NAATs for GBS do not characterize antibiotic susceptibility, which can inform further treatment in the case of a positive GBS result. Accordingly there is a need for improved testing methods characterized by a reduced turnaround time as compared to enrichment-based approaches. Further, there is a need for NAATs for detection of GBS that facilitate the characterization of antibiotic susceptibility.

In order to overcome the aforementioned drawbacks of enrichment based testing and existing NAATs, a novel approach was conceived of and developed for the identification of GBS as disclosed herein. In one aspect, an NAAT according to the present disclosure provides for direct from specimen testing without the need for enrichment. To achieve this, a number of potential markers were evaluated. For a marker to be useful for detecting in a direct from specimen sample without enrichment, it was anticipated that a relatively abundant nucleic acid could serve as a suitable target. Given that a typical microbial cell's total RNA pool is mainly composed of ribosomal RNA (rRNA), several rRNA components were evaluated, including the GBS 23SrRNA. To further improve confidence in the NAAT of the present example, the cfb gene, which encodes the Christie, Atkins, and Munch-Peterson (CAMP) factor and is recommended for use in identification of GBS by ASM, was included as an additional target.

With the goal of developing a dual-target NAAT for direct from specimen testing, primers and probes were designed and tested for the detection of both the cfb gene and the novel GBS 23S rRNA target. Results of a PCR assay for detecting GBS 23S rRNA and cfb in multiplex are shown in Table 7. The assay included three sets of oligonucleotides—each set including a forward primer, reverse primer, and detectable probe—with one set for detecting GBS 23S rRNA and two sets for detecting cfb. In one aspect, accurate detection of cfb according to the present example is achieved through the use of two sets of oligonucleotides inclusive for different GBS serotypes of interest, including GBS serotypes IV and V. Specifically, the oligonucleotides tested were forward primers GBS021 (SEQ ID NO: 22), GBS031 (SEQ ID NO: 28), and GBS039 (SEQ ID NO: 31), reverse primers GBS025 (SEQ ID NO: 24), GBS033 (SEQ ID NO: 29), and GBS041 (SEQ ID NO: 32), and probes GBS027 (SEQ ID NO: 26), GBS004 (SEQ ID NO: 04), and GBS059 (SEQ ID NO: 64). Templates for GBS or GBS 23S rRNA detection were purified synthetic target gene amplicons, with digital droplet PCR (ddPCR) corrected copy number.

TABLE 7
Mastermix

| | | |
|---|---|---|
| Test Conc. (cp/rxn) | Avg. Ct | Avg. RFI |

GBS 23S rRNA detection

| | | |
|---|---|---|
| 1.00E+04 | 29.56 | 23.98 |
| 1.00E+03 | 33.31 | 18.41 |
| 1.00E+02 | 36.79 | 21.82 |
| 1.00E+01 | 40.82 | 15.07 |
| 1.00E+00 | 42.68 | 17.19 |
| NTC | — | — |

cfb detection

| | | |
|---|---|---|
| 1.00E+04 | 27.14 | 22.59 |
| 1.00E+03 | 31.47 | 23.02 |
| 1.00E+02 | 35.20 | 24.51 |
| 1.00E+01 | 37.91 | 22.84 |
| 1.00E+00 | 41.12 | 16.54 |
| NTC | — | — |

As seen in Table 7, the primers and probes of the present example were successful in detecting both GBS 23S rRNA and cfb at all concentrations tested for the synthetic templates. However, an important consideration with GBS 23S rRNA designs is mitigation of background and/or cross-reactive signal. During testing of different primers and probes for detection of GBS 23S rRNA, it was noted that initial designs resulted in cross-reactivity (i.e., amplification and detection of) other closely related, non-GBS Streptococcus species including S. urinalis and S. thermophilus in Lim broth enriched clinical samples. As non-GBS Streptococcus species are known human commensals, the potential for such non-GBS Streptococcus species to be present in the samples was anticipated; however, it was surprisingly unexpected to observe cross-reactivity of the GBS 23S rRNA primers and probes to non-GBS Streptococcus species given that these oligonucleotides were designed to be highly specific for GBS.

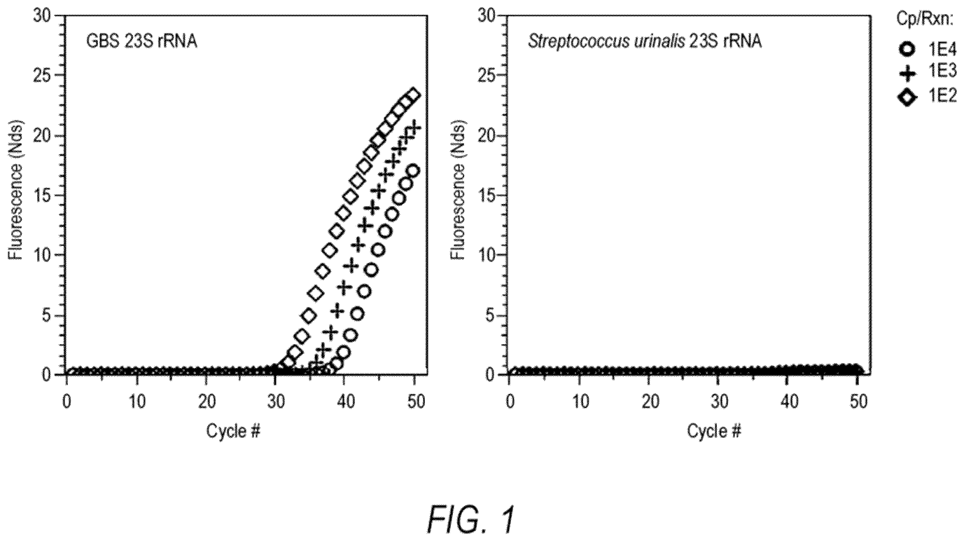

In order to overcome the unexpected cross-reactivity observed in the initial NAAT designs, a variety of modifications were considered for improving the specificity of the GBS 23S rRNA primers and probe. With reference to FIG. 1 and Table 8, an improved assay was developed that significantly reduces or eliminates cross-reactivity of the chosen 23S rRNA GBS target with 23S rRNA of closely related, non-GBS Streptococcus species including S. urinalis and S. thermophilus. This result was achieved, at least in part, through shifting the annealing site of the GBS 23S rRNA primers to reduce non-specific amplification resulting from homology at the 3′ end of the primers to non-GBS Streptococcus species and the design of a unique 23S rRNA probe including an N4-ethyl-dC modification. This probe modification was placed at the mismatch position between S. urinalis 23S rRNA and GBS 23S rRNA in the quencher region of the probe. The result was a highly decreased RFI with loss of called cycle threshold (Ct) value for S. urinalis and S. thermophilus, while maintaining Ct calls and RFI for GBS 23S rRNA. Assay results with GBS 23S rRNA forward and reverse primers GBS021 (SEQ ID NO: 22), and GBS025 (SEQ ID NO: 24), and S. urinalis exclusivity probe GBS069B (SEQ ID NO: 27) using GBS 23S and S. urinalis ddPCR quantified templates are shown in Table 8.

TABLE 8
Mastermix w/S. urinalis exclusive probe

| | | |
|---|---|---|
| Test Conc. (cp/rxn) | Avg. Ct | Avg. RFI |

GBS 23S rRNA detection

| | | |
|---|---|---|
| 1.00E+04 | 30.57 | 27.08 |
| 1.00E+03 | 34.29 | 25.76 |
| 1.00E+02 | 37.37 | 23.73 |
| 1.00E+01 | 41.27 | 23.63 |
| 1.00E+00 | 48.05 | 2.29 |
| NTC | — | — |

S. urinalis detection

| | | |
|---|---|---|
| 1.00E+04 | — | 1.15 |
| 1.00E+03 | — | 1.38 |
| 1.00E+02 | — | 1.36 |
| 1.00E+01 | — | 1.27 |
| 1.00E+00 | — | 1.2 |
| NTC | — | — |

In Tables 7 and 8, Test Conc. (cp/rxn) is the test concentration in units of copies per reaction, Avg Ct. is the average cycle threshold (i.e., the average number of cycles required for the fluorescence signal to cross a background fluorescence signal threshold), Avg RFI is the average relative fluorescence intensity, and NTC is no template control.

An alternative method to distinguish GBS 23S rRNA from other non-GBS Streptococcus signals involves employing two probes, each probe comprising one or more locked nucleic acid (LNA)-modified bases. In one aspect, a first probe, labeled with a dye, is designed to specifically bind to the GBS 23S rRNA target, while a second probe without a dye is designed to selectively bind to non-GBS targets. In this case, the second probe serves as a blocking probe to minimize nonspecific signals resulting from the presence of closely related, non-GBS Streptococcus species. Example oligonucleotides comprising LNA modifications listed in Table 6 include GBS084-GBS097 (SEQ ID NOs: 46-59), including blocking probes GBS085 (SEQ ID NO: 47), GBS0087 (SEQ ID NO: 49), GBS0089 (SEQ ID NO: 51), GBS0091 (SEQ ID NO: 53), GBS0093 (SEQ ID NO: 55), GBS0095 (SEQ ID NO: 57) and GBS0097 (SEQ ID NO: 59). Other approaches involving blocking probes that preferentially hybridize to non-target nucleic acid for which cross-reactivity is observed may be similarly applied.

The assays of the present example can be implemented on a real-time PCR device that supports two or more fluorescence detection channels. In one example, GBS 23S rRNA and cfb may be detected in a first channel and a generic internal control (GIC) may be detected in a second channel as shown in Table 9, which further indicates how the resulting data is interpreted based on the presence or absence of each of GBS 23S rRNA and cfb (23S/cfb) and GIC. It will be appreciated that for assays limited to the detection of GBS in a sample, the identification of either 23S rRNA or cfb is sufficient to characterize the sample as GBS-positive. Accordingly, for such assays, GBS 23S rRNA and cfb may be detected in the same channel. However, it may be useful in certain situations to detect GBS 23S rRNA and cfb in separate channels as discussed, for instance, in Example 6 below.

TABLE 9

| 23S/cfb | GIC | Interpretation |
|---|---|---|
| + | + | GBS |
| + | − | GBS |
| − | − | Invalid |
| − | + | No GBS |

### Example 6: Performance of Assay for Detection of GBS and Clindamycin Resistance Genes

Current NAATs for the detection and identification of GBS do not provide antimicrobial resistance information. Instead, a culture-based approach is necessary to obtain isolates to perform susceptibility testing. In one aspect, it is challenging to design a multiplex NAAT with the necessary sensitivity and specificity to confidently detect and characterize GBS in a sample, and in particular, a direct from specimen sample. The present example overcomes these and other challenges by providing primers and probes for use in an NAAT for the detection and identification of GBS in conjunction with the detection of one or more clindamycin resistance genes. The present NAAT may be used with both direct from specimen samples as well as with enriched samples.

Templates for clindamycin resistance targets ermB, ermTR, ermT, lsaC and lsaE were prepared from purified synthetic target gene amplicons with ddPCR corrected copy number. Nucleic acids extracted from cultured GBS strain cells, quantified through plate counting, served as templates for the GBS 23S rRNA and cfb genes.

Highly multiplexed results of sets of primers and probes for each i) GBS 23s rRNA, ii) the GBS-specific cfb gene, and iii) clindamycin resistance genes ermB, ermTR, ermT, lsaC and lsaE, are shown in Tables 10 and 11. The master mix included eight sets of oligonucleotides—each set including a forward primer, reverse primer, and detectable probe—with one set for detecting GBS 23S rRNA two sets for detecting cfb, and one set for each of the five different resistance targets (i.e., ermB, ermTR, ermT, lsaC and lsaE). Specifically, the oligonucleotides tested were forward primers GBS021 (SEQ ID NO: 22), GBS031 (SEQ ID NO: 28), GBS039 (SEQ ID NO: 31), ermB4.F (SEQ ID NO: 33), ermT6.F (SEQ ID NO: 13), ermTR_F_35_69TBB (SEQ ID NO: 38), SEG4091 (SEQ ID NO: 42), and SEGP3910 (SEQ ID NO: 19), reverse primers GBSO25 (SEQ ID NO: 24), GBS033 (SEQ ID NO: 29), GBSO41 (SEQ ID NO: 32), ermB_R_462_440 (SEQ ID NO: 34), ermT6.R (SEQ ID NO: 14), ermTR_R_203_171TBB (SEQ ID NO: 39), SEG4092 (SEQ ID NO: 43), and SEGP3911 (SEQ ID NO: 20) and probes GBS027 (SEQ ID NO: 26), GBS004 (SEQ ID NO: 04), GBS059 (SEQ ID NO: 64), GBS006 (SEQ ID NOs: 35 and 36), GBSO10 (SEQ ID NO: 37), ermTR8.JA270_10 (SEQ ID NOs: 40 and 41), GBS012 (SEQ ID NO: 44), and GBS015 (SEQ ID NO: 45).

TABLE 10

| | Resistance gene | | | | | | | | | |
|---|---|---|---|---|---|---|---|---|---|---|
| | ermB | | ermT | | ermTR | | lsaC | | IsaE | |
| Test Conc. | Avg | Avg | Avg | Avg | Avg | Avg | Avg | Avg | Avg | Avg |
| (cp/Rxn) | Ct. | RFI | Ct. | RFI | Ct. | RFI | Ct. | RFI | Ct. | RFI |
| 1.00E+04 | 25.52 | 44.44 | 26.76 | 6.28 | 27.51 | 48.66 | 27.67 | 4.77 | 25.97 | 3.64 |
| 1.00E+03 | 29.15 | 44.90 | 30.54 | 6.32 | 30.69 | 46.06 | 31.95 | 4.44 | 28.91 | 3.62 |
| 1.00E+02 | 32.56 | 44.29 | 35.05 | 6.24 | 34.43 | 45.20 | 34.81 | 3.85 | 32.31 | 3.58 |
| 1.00E+01 | 35.93 | 41.65 | 38.14 | 6.36 | 37.02 | 44.19 | 38.47 | 2.88 | 36.55 | 3.41 |
| 1.00E+00 | 38.34 | 48.95 | 40.87 | 7.40 | 40.23 | 23.45 | 42.35 | 1.63 | 40.14 | 2.99 |
| NTC | — | — | — | — | — | — | — | — | — | — |

TABLE 11

| | GBS strain | |
|---|---|---|
| Test Conc. | 23S | cfb |

| (CFU/Rxn) | Avg Ct. | Avg RFI | Avg Ct. | Avg RFI |
|---|---|---|---|---|
| 1.00E+04 | 19.42 | 5.89 | 27.20 | 46.25 |
| 1.00E+03 | 23.02 | 5.79 | 30.87 | 46.48 |
| 1.00E+02 | 25.62 | 5.79 | 33.43 | 45.60 |
| 1.00E+01 | 30.39 | 5.83 | 37.12 | 43.88 |
| 1.00E+00 | 33.84 | 5.72 | 41.05 | 36.24 |
| NTC | — | — | — | — |

In Tables 10 and 1, Test Conc. (CFU/Rxn) is the test concentration in units of cell forming units per reaction, Avg Ct. is the average cycle threshold (i.e., the average number of cycles required for the fluorescence signal to cross a background fluorescence signal threshold), Avg RFI is the average relative fluorescence intensity, and NTC is no template control.

The assays of the present example can be implemented on a real-time PCR device that supports two or more fluorescence detection channels. Preferably, the real-time PCR device supports at least three fluorescence detection channels. More preferably, the real-time PCR device supports at least four fluorescence detection channels. In the present example, Detecting and distinguishing GBS having one or more clindamycin resistance genes from other bacteria harboring clindamycin resistance genes can be achieved by assays separating GBS 23S rRNA and cfb targets in distinct channels as shown in Table 12, and rule based interpretation calling as shown in Table 13. In the example shown in Table 12, GBS 23S rRNA may be detected in a first channel (e.g., channel 3), cfb may be detected in a second channel (e.g., channel 1), the one or more clindamycin resistance genes may be detected in a third channel (e.g., channel 4), and a generic internal control (GIC) may be detected in a fourth channel (e.g., channel 5).

Probes GBS80-GBS83 (SEQ ID NOs: 60-63) include a Coumarin dye as the donor moiety and are designed to hybridize to and detect cfb. As cfb is present as a single DNA copy in the GBS genome, it represents a low copy target. Here, low copy means less than or equal to 5 copies per genome. Similarly, the clindamycin resistance genes are also found as either single or low copy chromosomal gene targets or on low copy plasmids. By determining the single target Ct for each of cfb and any resistance genes present in the sample, a ΔCt correlation between each of the resistance genes and cfb can be calculated in order to better inform if the level of resistance gene detected in a sample is similar to that of the GBS specific cfb gene. This information can then be utilized to supply more accurate information on if the given resistance signal was likely obtained from GBS versus another gram positive organisms.

TABLE 12

| Channel 1 | Channel 2 | Channel 3 | Channel 4 | Channel 5 |
|---|---|---|---|---|
| cfb | | 23S | ermB | GIC |
| | | | ermTR | |
| | | | ermT | |
| | | | lsaC | |
| | | | lsaE | |

TABLE 13

Interpretation

| 23S | cfb | ClinR | GIC | GBS | Resistance |
|---|---|---|---|---|---|
| + | + | − | + | + | − |
| + | − | − | + | + | − |
| − | + | − | + | + | − |
| + | + | + | + | + | +* |
| − | + | + | + | + | +* |
| + | − | + | + | + | + |
| − | − | + | + | − | + |
| − | − | − | + | − | + |
| + | − | − | − | + | Invalid |
| − | + | − | − | + | Invalid |
| − | − | + | − | Invalid | + |

With reference to Table 13, an asterisk (*) indicates that ΔCt between the Ct for cfb and the Ct for a given resistance gene can be utilized to further correlate resistance originating from GBS.

In one example, ΔCt is calculated as the difference between the called Ct for cfb (or another low copy GBS-specific gene such as the surface immunogenic protein encoding gene, sip, a glycosyl transferase protein encoding gene, and a lysR family protein encoding gene) in the sample (Ct_{GBS}) and the called Ct for a single target clindamycin resistance gene (Ct_{ClinR}) such as ermB, ermTR, ermT, lsaC andlsaE. This may be written as ΔCt=Ct_{GBS}−Ct_{ClinR}. If the absolute value of ΔCt is less than or equal to a defined threshold value (x), then it can be determined that the sample comprises GBS harboring the clindamycin resistance gene. In one aspect, when x is small (e.g., 2 or less), then the relative quantities of cfb (or another low copy GBS-specific gene) and the target clindamycin resistance gene can be considered to be similar or the same, and therefore likely to originate from the same organism. More particularly, it can be inferred that the GBS strain, as identified by cfb, is harboring the clindamycin resistance gene. By contrast, if x is large, then it is likely that identification of the clindamycin resistance gene is due to the presence of a second, non-GBS organism harboring the clindamycin resistance gene. The appropriate value for x can be determined empirically and may depend on the nature of the NAAT, including the choice of instrument, buffers, consumables, and the like. In one example, x is less than or equal to 2. In another example, x is less than or equal to 2.5. In another example, x is less than or equal to 3.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method for detecting Group B Streptococcus (GBS), the method comprising:
contacting a sample with a plurality of oligonucleotide primers for a defined set of targets to produce an amplification product comprising a representative nucleic acid for each of the targets present in the sample;
combining the amplification product with a detectable oligonucleotide probe for each of the targets; and
detecting the presence or absence of each of the representative nucleic acids in the amplification product,
wherein the presence or absence of each of the representative nucleic acids in the amplification product is indicative of the presence or absence in the sample of each of a GBS strain and a clindamycin resistance mechanism.

2. The method of claim 1, wherein the sample is selected from one of an enriched sample and a direct from specimen sample.

3. The method of claim 1, wherein the defined set of targets comprises: i) a GBS 23s ribosomal RNA gene (23s rRNA), ii) a GBS-specific gene, and iii) at least one clindamycin resistance gene.

4. The method of claim 3, wherein the GBS-specific gene is selected from the group consisting of CAMP-factor encoding gene (cfb), surface immunogenic protein encoding gene (sip), a glycosyl transferase protein encoding gene, and a lysR family protein encoding gene.

5. (canceled)

6. The method of claim 3, wherein the at least one clindamycin resistance gene is selected from the group consisting of ermTR, ermB, ermT, lsaC, and lsaE.

7. The method of claim 3, wherein the plurality of oligonucleotide primers comprises a set of oligonucleotide primers for amplifying at least a portion of each of targets, wherein:
the set of 23s rRNA oligonucleotide primers comprises at least one primer comprising a nucleic acid sequence of SEQ ID NOs: 22, 23 or 24;
the set of GBS-specific gene oligonucleotide primers comprises at least one primer comprising a nucleic acid sequence of SEQ ID NOs: 28, 29, 31, or 32; and
the at least one set of clindamycin resistance gene oligonucleotide primers is selected from the group consisting of:
a set of ermTR oligonucleotide primers comprising at least one primer comprising a nucleic acid sequence of SEQ ID NOs: 38 or 39,
a set of ermB oligonucleotide primers comprising at least one primer comprising a nucleic acid sequence of SEQ ID NOs: 33 or 34,
a set of ermT oligonucleotide primers comprising at least one primer comprising a nucleic acid sequence of SEQ ID NOs: 13 or 14,
a set of lsaC oligonucleotide primers comprising at least one primer comprising a nucleic acid sequence of SEQ ID NOs: 42 or 43, and
a set of lsaE oligonucleotide primers comprising at least one primer comprising a nucleic acid sequence of SEQ ID NOs: 19 or 20.

8. The method of claim 3, wherein:
the detectable oligonucleotide probe for 23s rRNA comprises a nucleic acid sequence of SEQ ID NO: 27 or a complement thereof;
the detectable oligonucleotide probe for the GBS-specific gene comprises a nucleic acid sequence of SEQ ID NO: 30 or 64 or a complement thereof; and
the detectable oligonucleotide probe for the at least one clindamycin resistance gene is selected from the group consisting of:
a detectable oligonucleotide probe for ermTR comprising a nucleic acid sequence of SEQ ID NOs: 40 or 41 or a complement thereof,
a detectable oligonucleotide probe for ermB comprising a nucleic acid sequence of SEQ ID NOs: 35 or 36 or a complement thereof,
a detectable oligonucleotide probe for ermT comprising a nucleic acid sequence of SEQ ID NO: 37 or a complement thereof,
a detectable oligonucleotide probe for lsaC comprising a nucleic acid sequence of SEQ ID NO: 44 or a complement thereof, and
a detectable oligonucleotide probe for lsaE comprising a nucleic acid sequence of SEQ ID NO: 45 or a complement thereof.

9-12. (canceled)

13. The method of claim 3, further comprising:
measuring a cycle threshold (Ct) for each of the GBS-specific gene (Ct_{GBS}) and the at least one clindamycin resistance gene (Ct_{ClinR});
calculating the difference between Ct_{GBS} and Ct_{ClinR} as ΔCt; and
identifying the sample as comprising GBS harboring the at least one clindamycin resistance gene when the absolute value of ΔCt is less than or equal to a threshold value (x).

14. The method of claim 13, wherein x≤2.

15. (canceled)

16. A method for detecting Group B Streptococcus (GBS), the method comprising:
contacting a sample with a plurality of oligonucleotide primers for a defined set of targets to produce an amplification product comprising a representative nucleic acid for each of the targets present in the sample;
combining the amplification product with a detectable oligonucleotide probe for each of the targets; and
detecting the presence or absence of each of the representative nucleic acids in the amplification product,
wherein the presence or absence of each of the representative nucleic acids in the amplification product is indicative of the presence or absence in the sample of a GBS strain; and
wherein the method distinguishes GBS from other species of Streptococcus.

17. The method of claim 16, wherein the sample is selected from one of an enriched sample and a direct from specimen sample.

18. The method of claim 16, wherein the defined set of targets comprises a GBS 23s ribosomal RNA gene (23s rRNA).

19. The method of claim 18, wherein the defined set of targets further comprises a GBS-specific gene, and wherein the GBS-specific gene is the CAMP-factor encoding gene (cfb).

20. (canceled)

21. The method of claim 16, wherein the method distinguishes GBS from at least one of Streptococcus urinalis, Streptococcus thermophilus, and Streptococcus anginosus.

22. The method of claim 16, wherein the defined set of targets further comprises at least one clindamycin resistance gene, and wherein the at least one clindamycin resistance gene is selected from the group consisting of ermTR, ermB, ermT, lsaC, and lsaE.

23. (canceled)

24. The method of claim 16, wherein the amplification product is further combined with a blocking oligonucleotide.

25-26. (canceled)

27. A method for detecting Group B Streptococcus (GBS), the method comprising:
contacting a sample with a plurality of oligonucleotide primers for a defined set of targets to produce an amplification product comprising a representative nucleic acid for each of the targets present in the sample;
combining the amplification product with a detectable oligonucleotide probe for each of the targets; and
detecting the presence or absence of each of the representative nucleic acids in the amplification product,
wherein the presence or absence of each of the representative nucleic acids in the amplification product is indicative of the presence or absence in the sample of a GBS strain; and
wherein the defined set of targets comprises a GBS 23s ribosomal RNA gene (23s rRNA).

28. (canceled)

29. The method of claim 27, wherein the defined set of targets further comprises at least one of a GBS-specific gene and a clindamycin resistance gene.

30. The method of claim 27, wherein the method distinguishes GBS from at least one of Streptococcus urinalis, Streptococcus thermophilus, and Streptococcus anginosus.

31. The method of claim 27, wherein the plurality of oligonucleotide primers comprises a set of oligonucleotide primers for amplifying at least a portion of each of targets, wherein the set of 23s rRNA oligonucleotide primers comprises at least one primer comprising a nucleic acid sequence of SEQ ID NOs: 22, 23 or 24, and wherein the detectable oligonucleotide probe for 23s rRNA comprises a nucleic acid sequence of SEQ ID NO: 27 or a complement thereof.

32-33. (canceled)
